(19) United States
(12) Patent Application Publication
(10) Pub. No.: US 2021/0181182 A1
(43) Pub. Date: Jun. 17, 2021

(54) METHOD FOR IDENTIFICATION OF PERTURBAGENS OF PARTICULAR BIOLOGICAL PROCESSES AND CELL SYSTEMS WITHIN LIVING CELLS

(71) Applicant: ATTAGENE, INC., Research Triangle Park, NC (US)

(72) Inventors: Sergei S. Makarov, Morrisville, NC (US); Alexander Vladimirovich Medvedev, Durham, NC (US)

(21) Appl. No.: 17/270,005

(22) PCT Filed: Aug. 20, 2019
(86) PCT No.: PCT/US2019/047365

§ 371 Date: Feb. 20, 2021

Related U.S. Application Data
(60) Provisional application No. 62/720,049, filed on Aug. 20, 2018.

(51) Int. Cl. G01N 33/50; G01N 33/68

(52) U.S. Cl. CPC G01N 33/5008; G01N 33/6803

(57) ABSTRACT

Methodology is described for identification of perturbagens of biological processes and cell systems through transcription factor activity profiling. The method is based on the unexpected finding that perturbagens of a particular biological process or a cell system within living cells produce an invariant transcription factor activity profile (TFAP), regardless of where and how said perturbagens interfere. Such invariant TFAPs have been identified for perturbagens of multiple biological processes and cell systems, including mitochondria, HDAC, and proteasome inhibitors; DNA damaging agents; cytoskeleton disruptors; and kinase inhibitors. The discovery of such invariant TFAP signatures opens a vast spectrum of applications, including drug discovery and repurposing of approved drugs, and provides a new ontological basis for characterization of biological properties of myriad chemical and biological compounds.

### CROSS-REFERENCE TO RELATED APPLICATION

The benefit under 35 USC §119 of U.S. Provisional Patent Application 62/720,049 filed Aug. 20, 2018 in the names of Sergei S. Makarov and Alexander Vladimirovich Medvedev for “Evaluating Biological Activity Of Compounds By Transcription Factor Activity Profiling” is hereby claimed. The disclosure of U.S. Provisional Patent Application 62/720,049 is hereby incorporated herein by reference, in its entirety, for all purposes.

### GOVERNMENT RIGHTS IN INVENTION

This invention was made with government support under R44GM125469 awarded by National Institutes of Health. The government has certain rights in the invention.

### BACKGROUND

### Field of the Invention

The present disclosure relates to the identification and analysis of biological activity of compounds by transcription factor activity profiling, and in a specific aspect relates to characterization and assessment of kinase inhibitors.

### Description of the Related Art

In order to predict the therapeutic use and toxicity of a compound, the biological activity of the compound must be known. This in turn requires finding the targets of the compound within a living cell, a task that is complex and difficult due to the myriad of cellular components that are present, and potential interactions of the compound with multiple targets.

The use of transcriptomics has become a favored strategy for making bioactivity assessments. Transcriptomics-based approaches characterize cell response to a compound by a “gene signature”, representing a list of differentially expressed genes. Mapping the differential genes to gene ontology provides useful information about the affected biological processes. Transcriptomic approaches have been widely used to annotate the biological activity of drugs and environmental chemicals. However, “backtracking” of transcriptomic gene expression changes to causal biological pathways requires highly complex computations and have met with only limited success. Particularly challenging in this regard are the assessments of poly-pharmacological compounds that interact with multiple targets.

It would therefore be a substantial advance in the art to provide a transcriptomic approach that enables rapid and reproducible identification of compounds with specified bioactivities among uncharacterized compounds, e.g., identification and characterization of multiple bioactivities of poly-pharmacological drugs.

### SUMMARY

The present disclosure relates to the use of transcription factor activity profiles to characterize responses to perturbations of biological pathways and systems, and associated methodology for identifying compounds with specific bioactivities among uncharacterized compounds, including assessment of multiple bioactivities of poly-pharmacological drugs.

The disclosure relates in one aspect to a method to identify perturbagens of particular biological processes and cell systems within living cells, said method comprising

exposing test cells to reference compounds from a group of known perturbagens of a particular biological process or a cell system within said cells;

evaluating the activity of transcription factors (TFs) within the exposed test cells resulting from said exposing to each reference compound;

identifying the consensus transcription factor activity profile (TFAP) for said group of reference compounds;

exposing test cells to an evaluated compound;

evaluating the activity of TFs within said test cells resulting from exposing the test cells to the evaluated compound,

calculating the similarity value of the TFAP of the evaluated compound to the consensus TFAP for the reference compounds, and

annotating the evaluated compound as a perturbagen of said biological process or said cell system if said similarity value exceeds a preset threshold value.

Various other aspects, features and embodiments of the disclosure will be more fully apparent from the ensuing description and appended claims.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIG. 1 illustrates the FACTORIAL™ assay enabling profiling TF responses to a chemical. Part A shows the flowchart of the FACTORIAL assay. Part B shows the TF endpoints of the FACTORIAL™ assay.

FIG. 2 shows the invariant TFAP signatures for inhibitors of the mitochondria function and proteasomal degradation. Part A shows representative TFAP signatures of mETC inhibitors. Part B shows representative TFAP signatures of UPP inhibitors. Part A and Part B tables show the similarity values r for the TFAPs of the individual perturbagens vs. the consensus TFAPs, calculated as a Pearson correlation coefficient. The radial graphs show TFAPs of perturbagens overlaying the consensus TFAPs. Note that the TF changes values are plotted in a log scale. Part C shows the TFAPs endpoints.

FIG. 3 shows the invariant TFAP signatures for HDAC inhibitors and cytoskeleton disruptors. Part A shows representative TFAP signatures of HDAC inhibitors. Part B shows representative TFAP signatures of MTDs. Part A and Part B tables show the similarity values r for the TFAPs of perturbagens at indicated concentrations vs. the consensus TFAPs, calculated as a Pearson correlation coefficient r. The radial graphs show TFAPs of perturbagens overlaying the consensus TFAPs. Part C shows the TFAPs endpoints.

FIG. 4 illustrates that low and high doses of DNA damaging agents produce distinct TFAP signatures. The Part A table shows the similarity values r for the TFAPs of perturbagens at indicated concentrations vs. the consensus clusters' TFAPs, calculated as a Pearson correlation coefficient. Parts B and C show the radial graphs and show representative TFAPs of perturbagens overlaying the consensus TFAPs. Part D shows the TFAP endpoints.

FIG. 5 illustrates that invariant TFAP signatures afford the identification of compounds with specified bioactivities. Part A shows the comparison of the TFAP-based prediction of mitochondria inhibitors with the data by a functional assay. Upper panel: Total of 2,793 ToxCast chemicals were assessed by the mitochondria membrane potential (MMP) and the FACTORIAL assays. Central panel: shows the recall of MMP-positive chemicals (circles) and the concordance rates (bars) for the two assays. Bottom panel: The scaled Venn diagram illustrates the relationship between the two assays. Part B illustrates that the TFAP dataset query by the consensus TFAPs of mETC, UPP and HDAC inhibitors had retrieved compounds with corresponding bioactivities, with the graphs showing the similarity of the retrieved and consensus signatures.

FIG. 6 depicts the assessment of the on-target and off-target activities of poly-pharmacological drugs by TF activity profiling. Part A shows that the TFAP signature transition with increased concentration indicates an influence of off-target drug activities. Part B shows that the TFAP signatures enable the identification of the on-target and off-target activities of glitazones. Left panel. The identical TFAPs for low-concentration glitazones reflect the on-target activity (PPAR activation). Center panel. The identical secondary TFAPs for pioglitazone and troglitazone at higher concentrations indicate mitochondria malfunction. Right panel. At the highest concentration (60 uM), the troglitazone TFAP is identical to that of hydrogen peroxide, indicating oxidative stress. Part C depicts the assessment of dose-response of the PPAR RTU to glitazones to determine the AC50 values for the on-target activity, wherein each data point is an average of 3 independent measurements.

FIG. 7 shows the effect of mETC inhibitors on the viability of assay cells (HepG2).

FIG. 8 shows the effect of UPP inhibitors on the viability of assay cells and the common TFAP signatures for UPP inhibitors in HEK293 and MCF-7 cells.

FIG. 9 shows common TFAP signatures for HDAC inhibitors in HEK293 and MCF-7 cells. and the effect of HDAC inhibitors on the viability of assay cells.

FIG. 10 shows the effect of microtubule polymerization disruptors (MTDs) on the viability of assay cells.

FIG. 11 shows the effect of microtubule DNA damaging agents on the viability of assay cells.

FIG. 12 depicts an alternative presentation of TF responses to perturbagens as a heatmap.

FIG. 13 shows the list of known mitochondria disruptors with a high (r>0.800) TFAP similarity to the mETC TFAP that were scored negative by the MMP assay.

FIG. 14 in panels A-D shows invariant TFAP signatures in a radial graph format for HepG2 assay cells treated with the specified kinase inhibitors, wherein each of the 46 axes of the radiograph shows induced changes in activity of a particular transcription factor on a log scale, wherein a value of 1.0 indicates no changes in transcription factor activity, wherein the consensus TFAP signatures for each kinase class are average signatures of the indicated specific kinase inhibitor, showing the similarity of the individual kinase inhibitors' signatures versus the consensus signatures.

FIG. 15 shows that there was the main cluster A, containing signatures for most (6 out of 8) Akt inhibitors, and minor clusters (B and C), each containing a few signatures.

FIG. 16 illustrates the evaluation of inhibitors of mTOR, CDK, RAF, ERK, MEK, and Aurora showed that each kinase had its own, distinct invariant PKI signature.

### DETAILED DESCRIPTION

The present disclosure reflects the finding that transcription factor activity profiles (TFAPs) reveal invariant signatures of perturbed biological pathways and cellular systems, thereby enabling assessment of bioactivity of compounds by assessing activity of transcription factors that regulate gene expression.

In accordance with the present disclosure, the TFAPs are generated by use of a multiplex reporter system assay such as that commercially available from Attagene, Inc. under the trademark FACTORIAL, which uses a library of transcription factor-specific reporters and quantitative analysis to generate TFAPs, as more fully described in: U.S. Patent Application Publication 2010/0009348 of Sergei Romanov, et al. for “Methods and Constructs for Analyzing Biological Activities of Biological Specimens and Determining States of Organism”; Romanov et al., Nature Methods 5(3):253-60 (2008); and Sergei Makarov, et al. U.S. Patent Application Publication 2016/0333428 for “Multiplexing Transcription Factor Reporter Protein Assay Process and System”, the disclosures of which are hereby incorporated herein by reference.

The term “perturbagens”, as used herein, broadly denotes compounds that affect particular biological pathways to produce responsive transcription factor activity, and thereby produce distinct TFAP signatures. The present inventors have unexpectedly found that perturbagens of the same biological pathway all produce invariant TFAPs, regardless of where or how they interfere with such pathway. As a result of such discovery, the present inventors have demonstrated invariant TFAPs for a large spectrum of compounds, e.g., mitochondrial, histone deacetylase (HDAC), and ubiquitin/proteasome pathway inhibitors; cytoskeleton disruptors; and DNA-damaging agents. Use of the invariant TFAP signatures has been found to enable direct and reproducible identification of compounds with specified bioactivities among uncharacterized compounds, and facilitates assessment of multiple bioactivities of poly-pharmacological drugs. Accordingly, transcription factor activity profiling in accordance with the present disclosure affords a ready assessment of bioactivity in response to exposure to compounds, through the identification of perturbed biological pathways resulting from such exposure.

The methodology of the present disclosure may be implemented in any of the implementations variously described herein.

In one embodiment, the disclosure relates to a method to identify perturbagens of particular biological processes and cell systems within living cells, said method comprising

exposing test cells to reference compounds from a group of known perturbagens of a particular biological process or a cell system within said cells;

evaluating the activity of transcription factors (TFs) within the exposed test cells resulting from said exposing to each reference compound;

identifying the consensus transcription factor activity profile (TFAP) for said group of reference compounds;

exposing test cells to an evaluated compound;

evaluating the activity of TFs within said test cells resulting from exposing the test cells to the evaluated compound,

calculating the similarity value of the TFAP of the evaluated compound to the consensus TFAP for the reference compounds, and

annotating the evaluated compound as a perturbagen of said biological process or said cell system if said similarity value exceeds a preset threshold value.

In such method, the perturbagen may be an inhibitor, an activator, or a disruptor of a biological process or cell system. The perturbagen may for example be an inhibitor of mitochondria function, ubiquitin-proteasome-mediated protein degradation process, histone deacetylation, protein phosphorylation, protein dephosphorylation, lipid phosphorylation, or lipid dephosphorylation. In various embodiments, the perturbagen may be a DNA damaging agent, a cytoskeleton disruptor, or an inducer of proteotoxic shock. In other embodiments, the perturbagen may be an inhibitor of an Akt kinase, an mTOR kinase, a Mek kinase, a Raf kinase, a ERK kinase, an Aurora kinase, a cyclin dependent kinase, or a phosphodiesterase inhibitor.

The method broadly described above may be carried out, wherein the group of known reference compounds comprises at least two compounds.

In various embodiments of the disclosure, the TF activity profile comprises activity values for at least 3 TFs, and may for example comprise TFs that are selected from the following group: NF-kappaB, AP-1, myc, HIF-1a, p53, PXR, MTF-1, HSF-1, beta-catenin/TCF. FThe method of the disclosure may be conducted, wherein TF activity is determined using an assay permitting quantitative assessment of TF activity. The assay may for example be a DNA binding assay, a gel-shift assay, a reporter gene assay, or a transcription-based homogeneous reporter system assay. Preferably, the assay is a transcription-based homogeneous reporter system assay (FACTORIAL™ assay).

In the method of the disclosure, the test cells may be of any suitable type, and may for example be mammalian cells, e.g., human cells, such as HepG2 cells.

The method of the disclosure may be carried out, wherein said TFAP similarity value for TFAPs is a Pearson correlation coefficient or a Euclidean distance, as a pairwise assessment. In various embodiments, the TFAP similarity threshold is >0.5; >0.6; >0.7; >0.8; >0.9; or≥0.95, or other suitable value, minimum, or limit.

The method of the disclosure may be carried out, wherein the consensus TFAPs for the group of reference compounds is identified using cluster analysis of TFAPs for individual perturbagens.

In the method of the disclosure, the evaluated compound may be of any suitable type and may for example be or comprise a chemical, a mix of chemicals, an RNA molecule, a DNA molecule, a peptide, a protein, or an antibody. In various embodiments the evaluated compound may be a biological specimen. The evaluated biological specimen may be of any suitable type, e.g., a saliva, a urine, a feces, a blood serum, a blood plasma, a cell extract, or a tissue extract.

The method of the disclosure may be carried out with the test cells being exposed to different concentrations of reference perturbagens. Generally, the test cells may be exposed to the reference compounds for any suitable predetermined time, e.g., more than 0.5 hrs, more than 1 hrs, more than 3 hrs, more than 12 hrs, more than 24 hrs, more than 48 hrs, or more than 72 hrs.

The method of the present disclosure as variously described above may be conducted, in which the TFAP similarity value is calculated using a computer program. More generally, the method may be conducted wherein at least one of the method steps is a computer-implemented operation.

The present disclosure provides a methodology for characterization of biological activity resulting from exposure to evaluated compounds, which involves assessing responses of cellular signal transaction pathways that regulate gene expression, to evaluate activity of transcription factors that connect the signaling pathways with the regulated genes. Transcription factors are proteins that bind specific sequences within target genes, thereby modulating transcription, so that the description factor activity profile captures the signals that regulate gene expression.

Many transcription factor families that mediate cell responses to xenobiotics and various stress stimuli are well characterized, but less is known about how an interplay of multiple transcription factors coordinates gene expression. As transcription factor activity is regulated by various posttranslational modifications of preexistent transcription factor proteins, transcription factor activity may not correlate with transcription factor protein content. This necessitates the use of functional transcription factor assays, e.g., reporter gene assays. However, conventional transcription factor assays are not suited for assessing multiple TFs. Thus, multiplex reporter transcription factor assay systems such as the aforementioned FACTORIAL™ assay system, which enables parallel assessment of multiple transcription factors in a single well of cells, is usefully employed to analyze transcription factor responses to compounds, e.g., environmental chemicals and pharmaceutical agents, in accordance with the present disclosure.

In various embodiments of the present disclosure, the multiplex reporter transcription factor assay system is utilized to evaluate perturbations of biological pathways and cell systems in human cells. By such technique, cell responses to compounds are characterized by quantitative multi-endpoint signature, the transcription factor activity profile (TFAP). A major advantage of the transcription factor activity profiling approach is that it generates simple quantitative signatures that provide clear quantitative metrics to compare the bioactivity of compounds.

The methodology of the present disclosure, in its various implementations as described herein, is based on the present inventors' observations that: (1) perturbations of biological pathways/cell systems produce distinct characteristic TFAP signatures; (2) perturbagens of the same biological pathway elicit an invariant TFAP regardless of where or how they interfere; (3) the invariant TFAPs enable the identification of compounds with the specified bioactivities among uncharacterized chemicals; and (4) the TFAPs enable assessing multiple biological activities of polypharmacological compounds.

The methodology of the present disclosure may be usefully employed in the drug development process. By assessing the TFAP signatures of a compound, one can identify its potential therapeutic uses and forecast its toxicity. By detecting the transformations of TFAP signatures with concentration, one can compare the off-target activities of drug candidates and the concentration windows in which the primary activity dominates. This in turn enables streamlined solutions for hit-to lead selection of potentially useful therapeutic agents. Moreover, the TFAP signatures provide insights into drug polypharmacology. While unintended polypharmacology can compromise patient safety, the drugs that modulate multiple disease-relevant targets can be unprecedentedly efficacious. In this respect, the TFAP signatures enable distinguishing between the unwanted and desirable polypharmacology. Further, the TFAP assessment enables a systematic and straightforward approach to the identification of new indications for approved drugs.

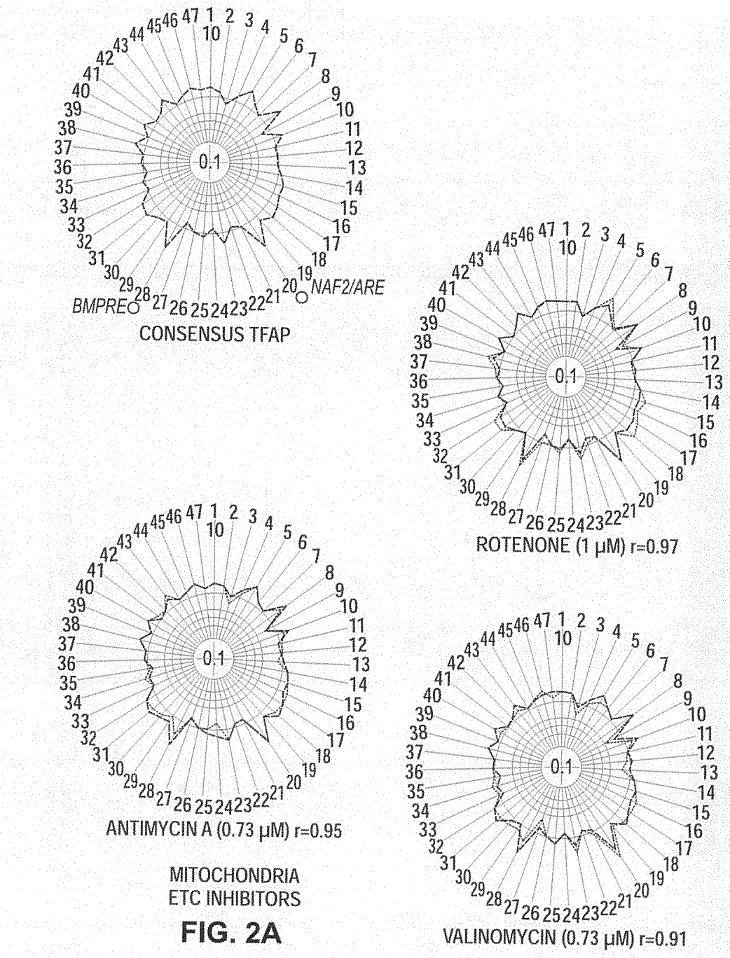

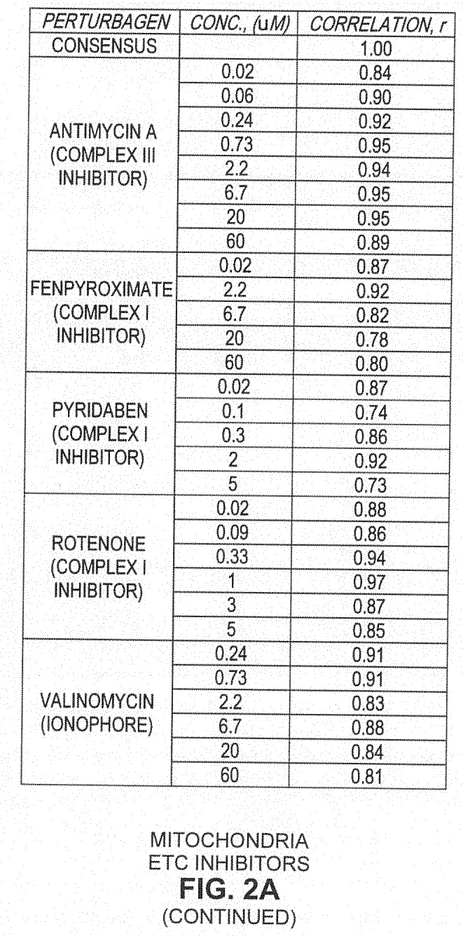

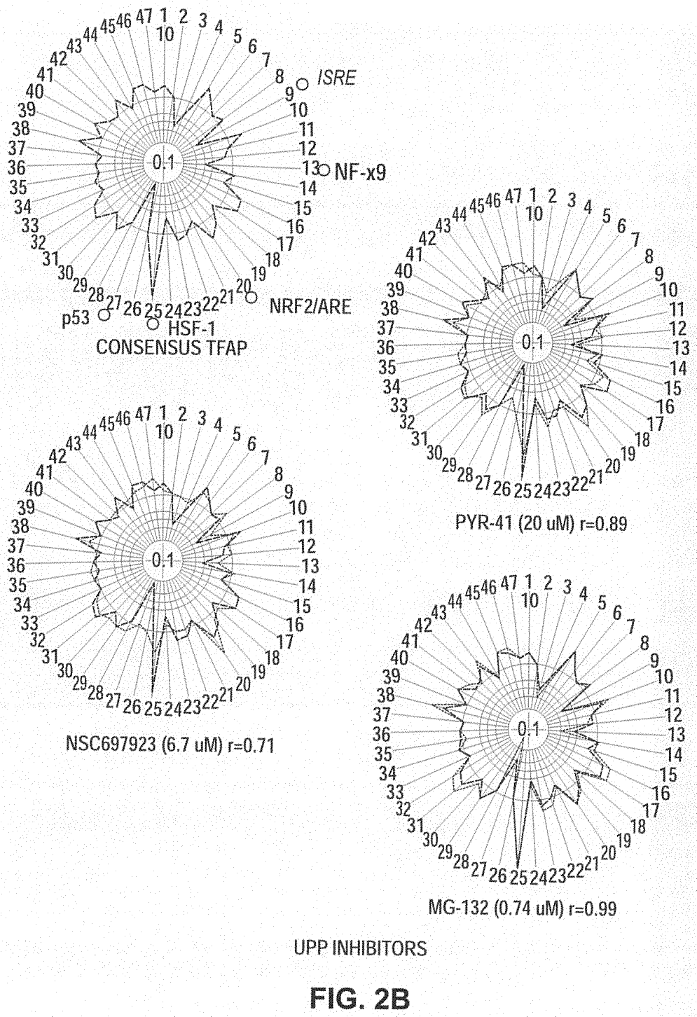

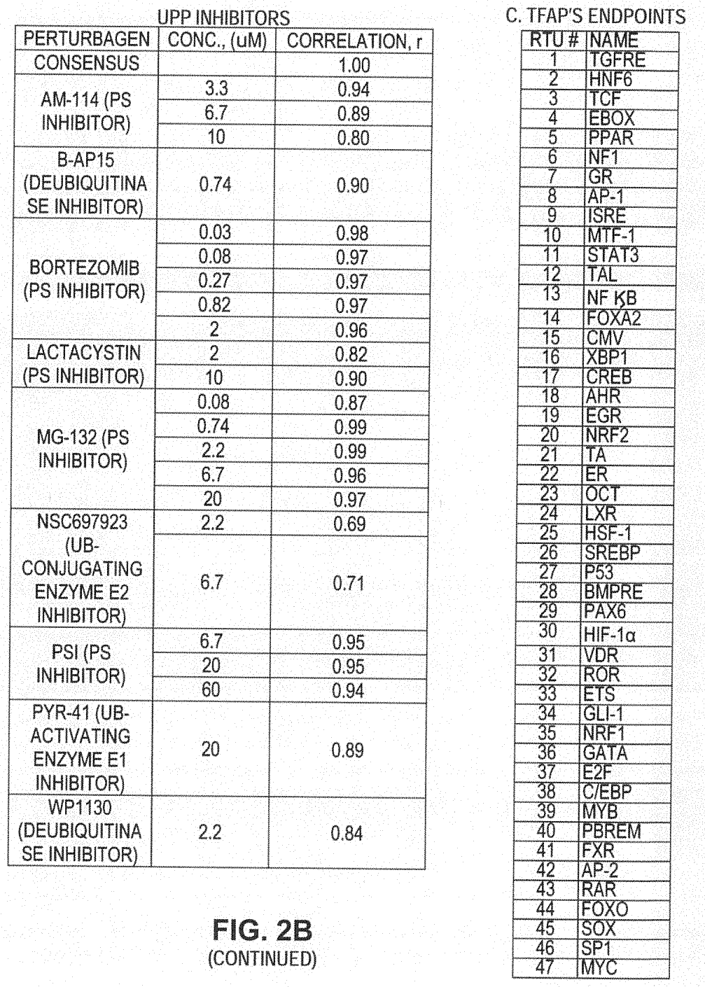

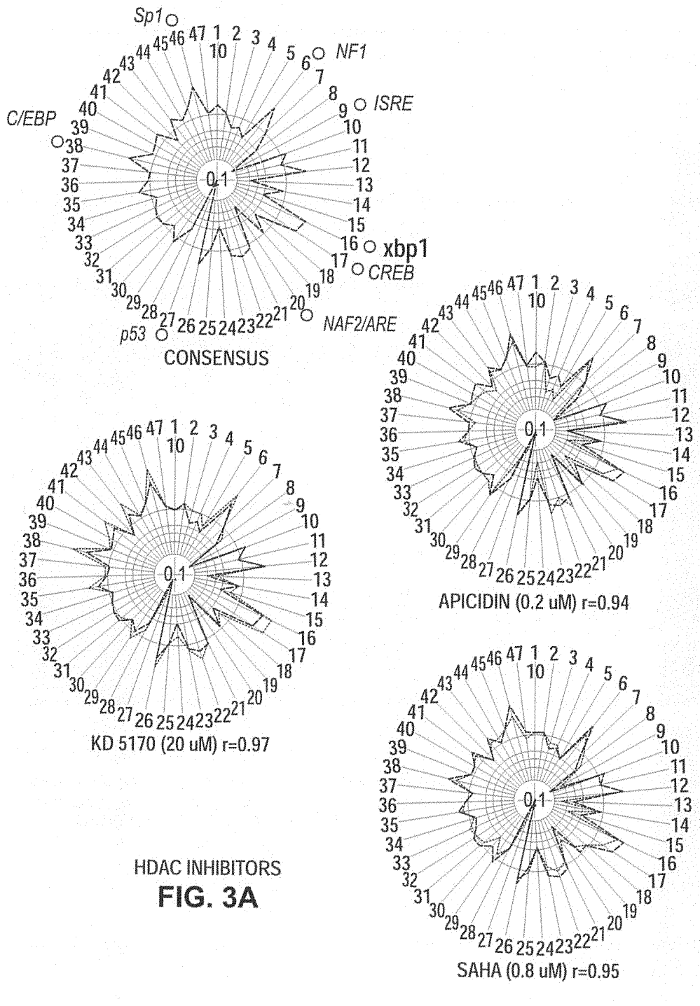

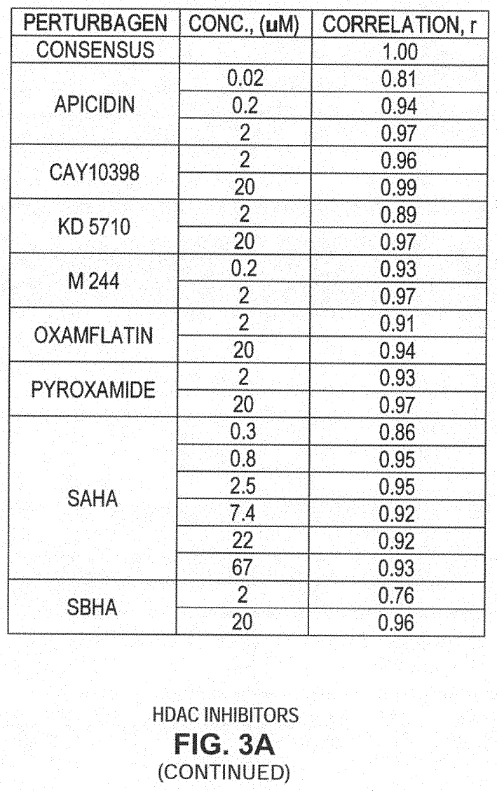

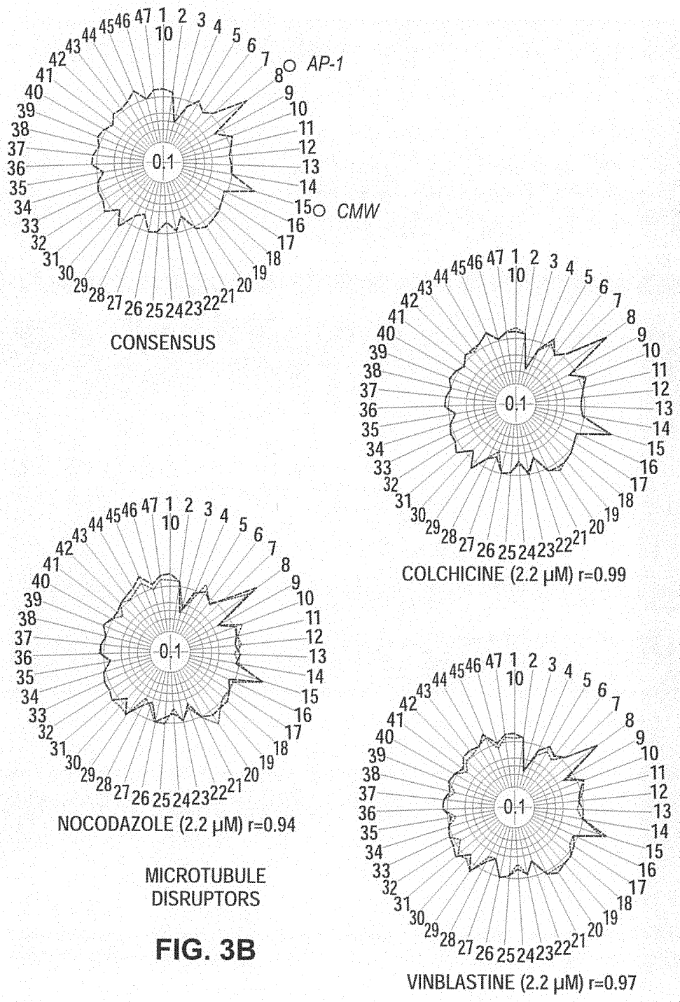

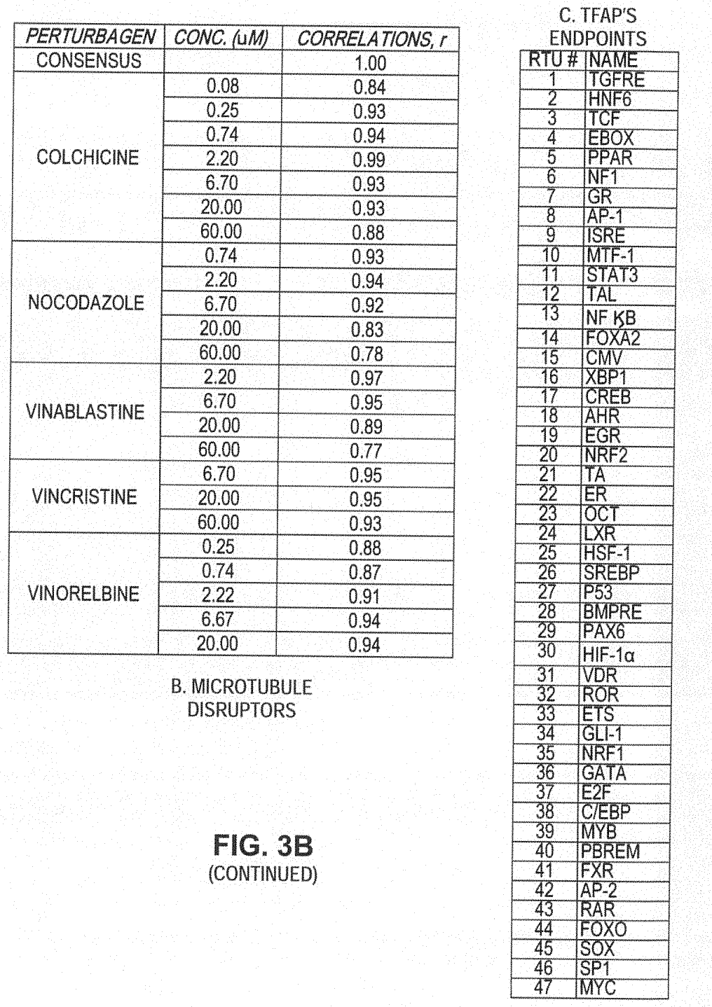

The finding of the invariant TFAPs indicates that disruptions of biological pathways and cell systems cause coordinated changes of transcription factor activity, which in turn implies an existence of specific response programs. Since cellular systems operate under permanent pressures of environmental and internal stresses, mechanisms are necessary present in such systems to cope with possible malfunctions. Some of these mechanisms have been described, such as the mitochondrial retrograde signaling that alters nuclear gene expression to adapt to mitochondria malfunctions. Another example is the “survival response” that coordinates changes in gene expression to accommodate the global epigenomic disruption by HDAC inhibitors. In this regard, the invariant TFAP signatures for mETC and HDAC perturbagens (shown in FIG. 2, Part A and FIG. 3, Part A, described hereinafter in further detail) epitomize the signals that regulate these programs.

The present inventors have identified invariant TFAPs for a diversity of biological pathways and pathway-modulating perturbagens, including mETC, UPP and HDAC inhibitors, cytoskeleton disruptors and DNA-damaging agents, as representative of the general existence of invariant signatures for perturbagens of other biological pathways and systems. The methodology of the present disclosure enables the generalized identification and characterization of perturbagens of biological pathways and systems, with the invariant signatures providing a new ontological basis for assessing bioactivity of myriad perturbagenic species.

The following description illustrates the methodology of the present disclosure, in specific embodiments and examples thereof.

Obtaining TFAP Signatures by the FACTORIAL™ Assay.

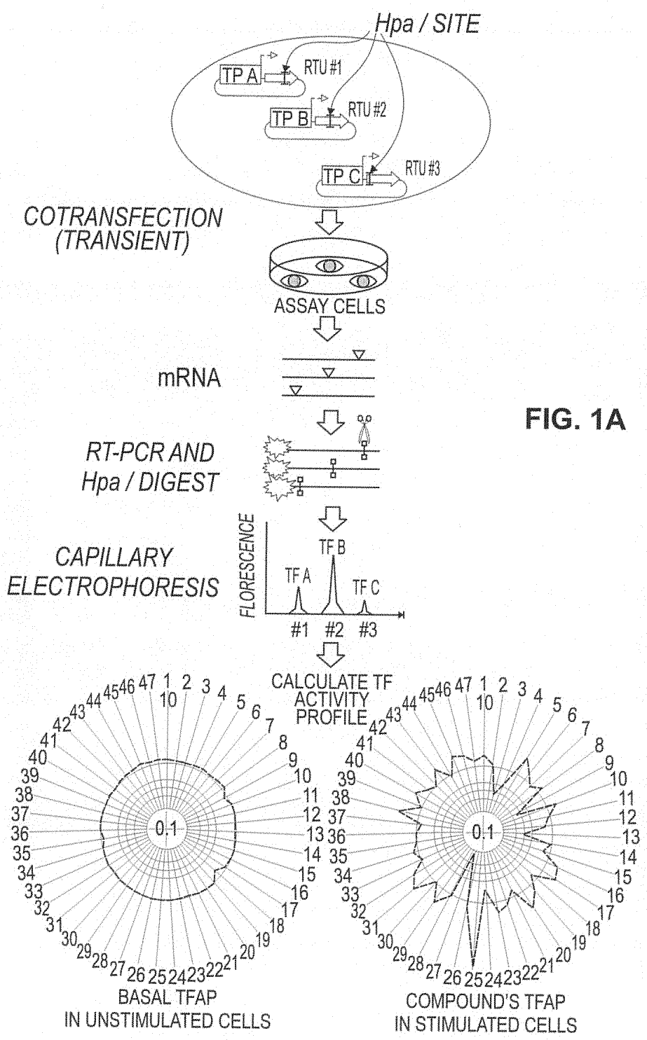

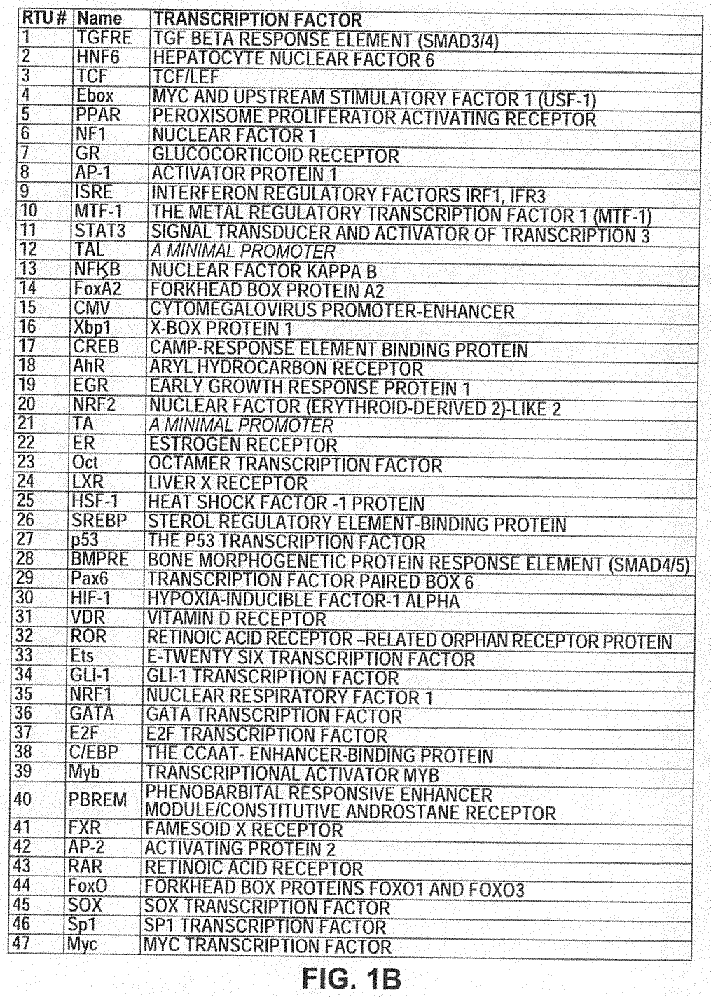

Using the FACTORIAL™ assay, TFAP signatures are obtained for perturbagens of various cell systems and biological pathways in human cells. The FACTORIAL™ reporter system comprises a set of TF-responsive reporter constructs called reporter transcription units (RTUs). Each RTU has a TF-specific promoter linked to a reporter sequence. Being transfected into assay cells, the RTUs produce reporter RNAs proportionate to the activity of their promoters. Thus, the activity of TFs by profiling the reporter transcripts can be assessed (FIG. 1 in Part A). Importantly, owing to a rapid turnover of reporter RNAs, this approach enables detecting not only activated, but also inhibited TFs.

To ensure equal detection efficacy for the TFs, a ‘homogeneous’ detection approach is employed. In this approach, all RTUs have identical reporter sequences tagged by a unique endonuclease site (HpaI) at a distinct position. The detection process entails RT-PCR amplification of the reporter transcripts, followed by fluorescent labeling and HpaI restriction. The labeled DNA fragments of predefined lengths are resolved by capillary electrophoresis (CE), producing the fluorescence profile that provides information about RTUs' activity (FIG. 1 in Part A). The homogeneous detection approach enables robust and reproducible TF assessments.

The FACTORIAL™ assay employed in this illustrative example comprised 47 RTUs for TFs that mediated responses to a variety of stress stimuli and xenobiotics (NF-kB, p53, AP-1, HIF-1a, HSF-1, etc.) (FIG. 1 in Part B). The RTU promoters contained one or multiple copies of TF binding sites for specified TF families. The reporter system also contained the RTUs with minimal TATA and TAL promoters and a cytomegalovirus (CMV) promoter.

Because chemicals have different cellular pharmacokinetics, the TFAP signatures were obtained after a prolonged (24-hr) exposure. The TFAP signatures are presented in a radial graph format with 47 axes showing the stimulus-induced TF activity changes. By the definition, the baseline TFAP in unstimulated cells is a perfect circle with the R=1.0 (FIG. 1 in Part A). To assess the similarity of two TFAPs, Pearson correlation coefficients are employed. The probability that two random 47-endpoint signatures have the similarity r>0.7 is less than 10⁻⁷; thus it is presumed that two compounds elicited identical responses if their signatures' similarity was above 0.7.

The Invariant TFAP of Mitochondria Inhibitors.

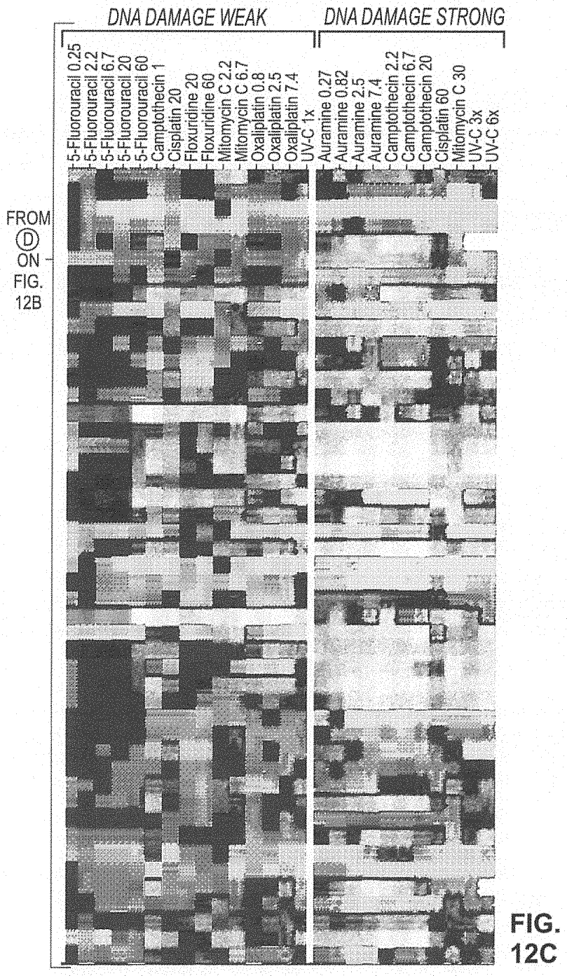

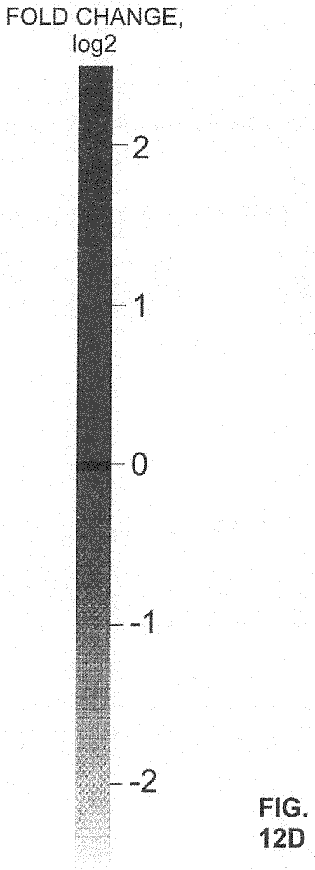

In one set of experiments, TFAPs of inhibitors of the mitochondrial electron transport chain (mETC) in human hepatocytic HepG2 cells were assessed. The ETC comprises functionally linked protein complexes within the internal mitochondrial membrane that transfer electrons from electron donors to oxygen. This process creates a proton gradient on the membrane that is converted into ATP production. A panel of specific inhibitors of different mETC complexes was evaluated, including rotenone, pyridaben, and fenpyroximate (complex I inhibitors); antimycin A (a complex III inhibitor); and an ionophore valinomycin. These chemicals were assessed at multiple concentrations. To compare multiple TFAPs, a clustering algorithm was developed that calculated an average (consensus) TFAP for the multiple chemicals, as described more fully below in Methods. The clustering of mETC inhibitors' TFAPs at r=0.7 revealed a single cluster. The consensus signature had a high similarity (r≥0.8) with the TFAPs of individual mETC inhibitors in a broad range of concentrations (FIG. 2 in Part A). For an alternative presentation of the TFAPs, a heatmap is shown in FIG. 12). The consensus mETC TFAP comprised multiple TF responses, including activation of oxidative stress-responsive NRF2/ARE, which was consistent with published data.

HepG2 cells produce sufficient amounts of ATP via glycolysis to survive fully anaerobic conditions, and none of mETC inhibitors caused cell death. Therefore, mETC perturbagens produced an invariant TFAP signature irrespective of their structural dissimilarity and their targets within the pathway.

The Invariant TFAP of Inhibitors of the Ubiquitin/Proteasome Protein Degradation Pathway.

The ubiquitin (Ub)/proteasome pathway (UPP) carries out regulated degradation of cellular proteins. This process involves a consecutive action of a Ub-activating enzyme (E1), a Ub-conjugating enzyme (E2), and a substrate-specific Ub ligase (E3) that attaches Ub residues to substrate proteins, targeting them to degradation by the 26S proteasome (PS). Using the FACTORIAL™ assay in HepG2 cells, TFAPs were obtained for a panel of chemicals specifically inhibiting different nodes of the UPP pathway, including PYR-41 (an E1 inhibitor with no activity at E2); NSC 697923 (a selective inhibitor of the E2 ubiquitin conjugating enzyme UBE2N); deubiquitinase inhibitors b-AP15 and WP1130; and five PS inhibitors, including lactacystin (an organic compound naturally synthesized by bacteria of the genus Streptomyces); peptide aldehydes MG132 and PSI, a boronic chalcone derivative AM114 and a peptide boronate bortezomib.

Clustering these signatures revealed a single cluster with the consensus TFAP signature that had a high similarity (r≥0.7) with TFAPs of individual UPP inhibitors in a broad range of concentrations (FIG. 2, in Part B). The consensus TFAP comprised multiple TF responses, including an activation of the heat shock factor 1 (HSF-1), NRF2/antioxidant response element (ARE) RTUs and inhibition of NF-kB RTU, that were in agreement with published data. Representative signatures of the UPP perturbagens are also shown as a heatmap in Fig. S6.

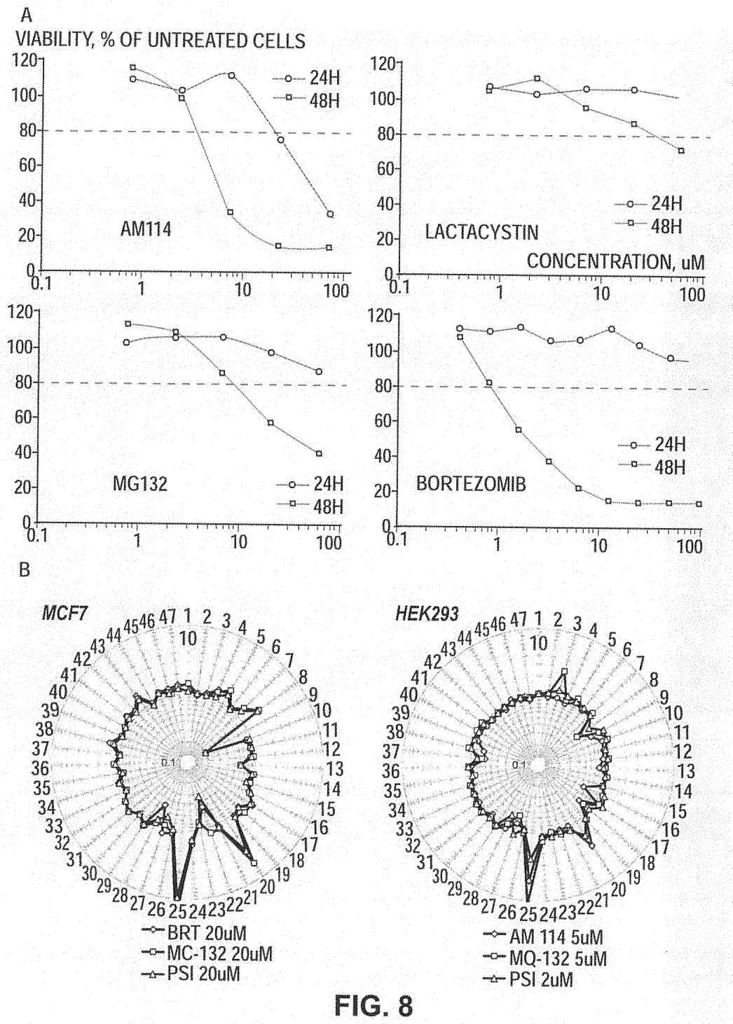

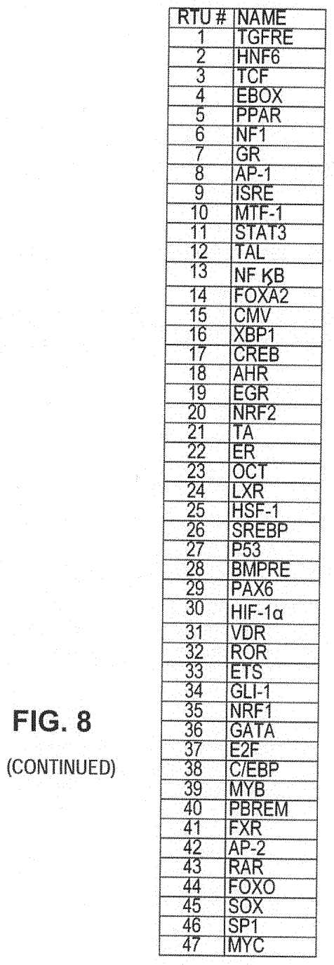

Since UPP inhibitors can cause cell death, cell viability was monitored. At the end of 24 hours incubation and at concentrations used, none of UPP inhibitors caused cell death. Some UPP inhibitors induced cell death after a prolonged (48 hour) incubation (FIG. 8, Part A). Thus, the invariant TFAP of UPP inhibitors cannot be explained by cell death.

The FACTORIAL™ assay in other cell types (mammary epithelial MCF-7 and kidney epithelial HEK293 cells) showed that different UPP inhibitors also produced common, yet cell type-specific, TFAP signatures (FIG. 8, Part B). The common TF responses that were observed in all cell types were the activation of HSF-1 and NRF2/ARE RTUs, while other TF responses varied among cell lines. Therefore, different UPP perturbations produced invariant TFAP signatures in different cell types, irrespective of perturbagens structure, their targets within the pathway, and their effects on cell viability.

The Invariant TFAP of HDAC Inhibitors.

Histone acetyl-transferases (HAT) and histone deacetylases (HDAC) are enzymes that catalyze the acetylation/deacetylation of histones and cellular proteins, which alters gene expression. Using the FACTORIAL™ assay in HepG2 cells, a panel of structurally diverse HDAC inhibitors was evaluated, including suberoylanilide hydroxamic acid (SAHA), suberohydroxamic acid (SBHA), CAY10398, M344, oxamflatin, pyroxamide, a cyclic peptide apicidin, and a mercaptoketone-based KD5170. The clustering revealed a single TFAP cluster with a consensus TFAP that had a high similarity (r≥0.8) with TFAPs of individual HDAC inhibitors at multiple concentrations (FIG. 3 in Part A). Representative TFAPs for HDAC inhibitors are also shown by the Fig. S6 heatmap.

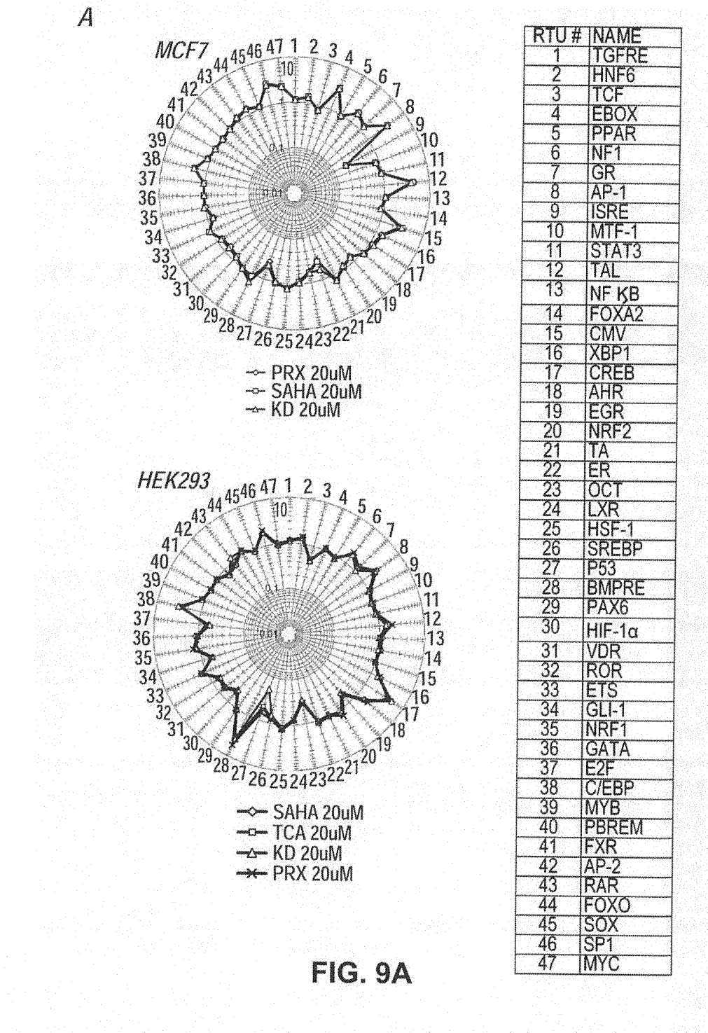

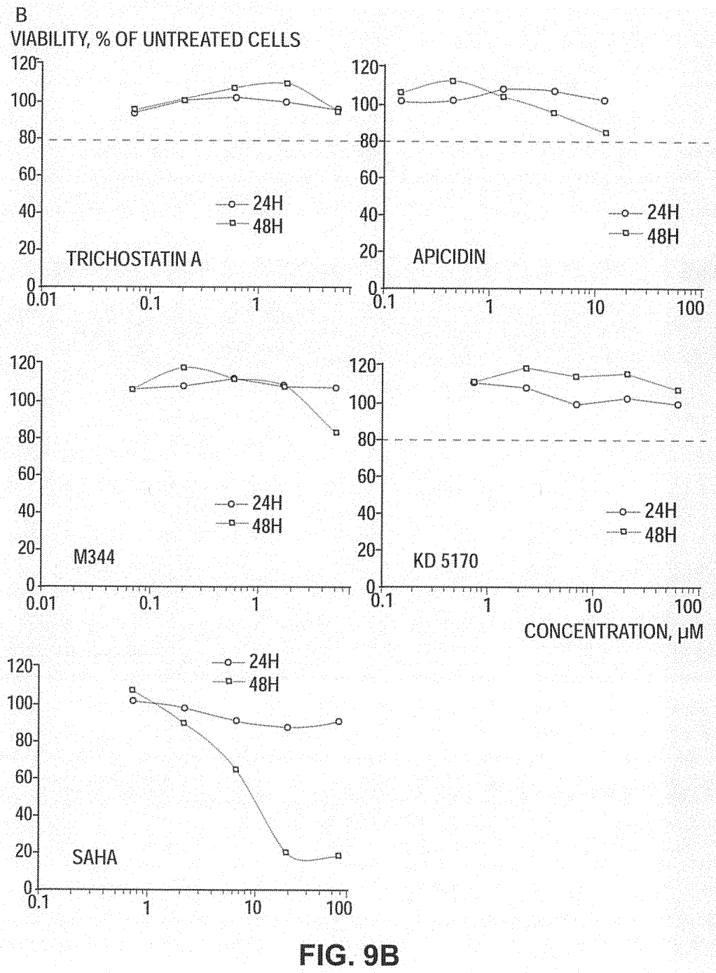

The FACTORIAL™ assay in HEK293 and in MCF-7 cells also revealed invariant, albeit cell type-specific signatures in these cells (FIG. 9, in Part A). After a 24 hour incubation, none of the HDAC inhibitors caused cell death (FIG. 9, in Part B). Thus, structurally dissimilar HDAC inhibitors produced invariant TFAPs in different cell types.

The Invariant TFAP of Cytoskeleton Disruptors.

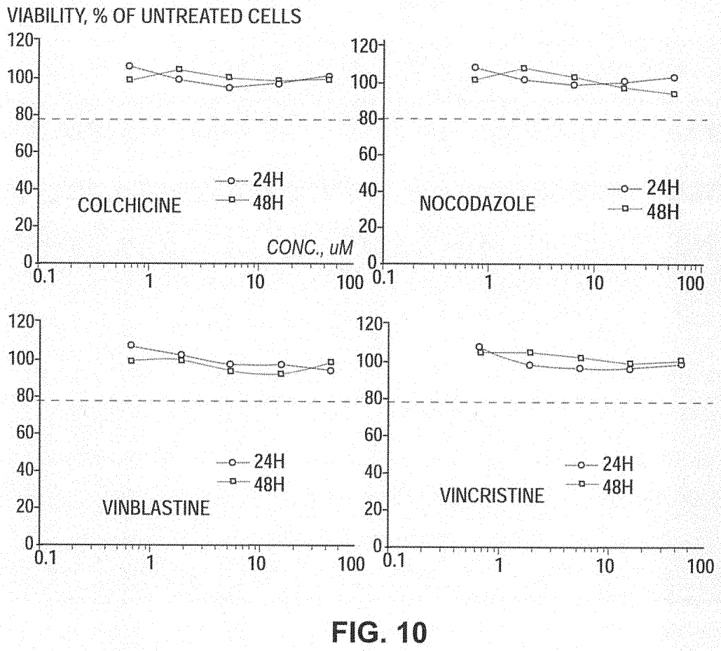

Microtubules are tubular polymers of tubulin that, along with microfilaments and intermediate filaments, constitute cytoskeleton of eukaryotic cells. Microtubules are dynamic structures whose length is regulated by the polymerization and depolymerization of tubulin. Using the FACTORIAL™ assay in HepG2 cells, a panel of structurally dissimilar microtubule polymerization disruptors (MTDs) was evaluated, including colchicine, nocodazole, vinblastine, vincristine, and vinorelbine. Cluster analysis revealed a single cluster with an invariant TFAP signature that had a high similarity to TFAPs of individual MTDs at multiple concentrations (r≥0.8) (FIG. 3 in Part B). The most prominent TF responses of the consensus TFAP were an upregulation of AP-1 and CMV RTUs and a downregulation of TCF/beta-catenin and estrogen response element (ERE) RTUs. These responses were in agreement with published data. Representative TFAPs of MTDs are also shown by the FIG. 12 as a heatmap. At the end of 24 hours incubation and at concentrations used, none of MTDs inhibited cell viability (FIG. 10). Therefore, structurally dissimilar microtubule polymerization disruptors produced an invariant TFAP.

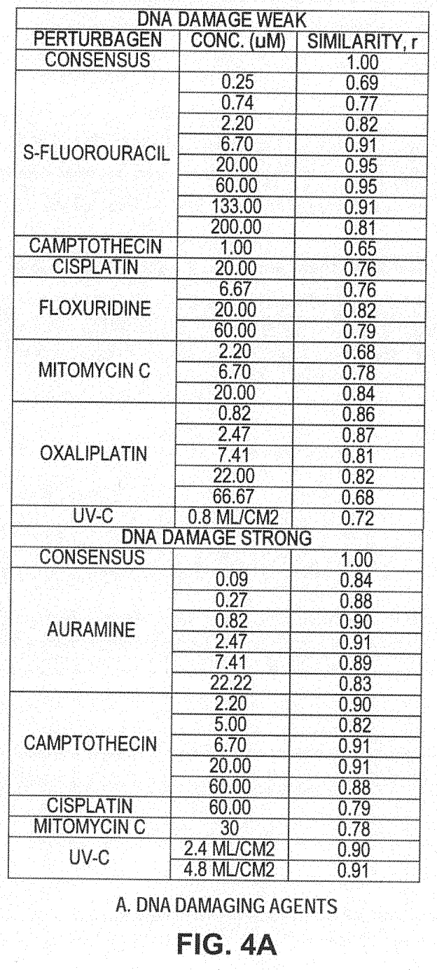

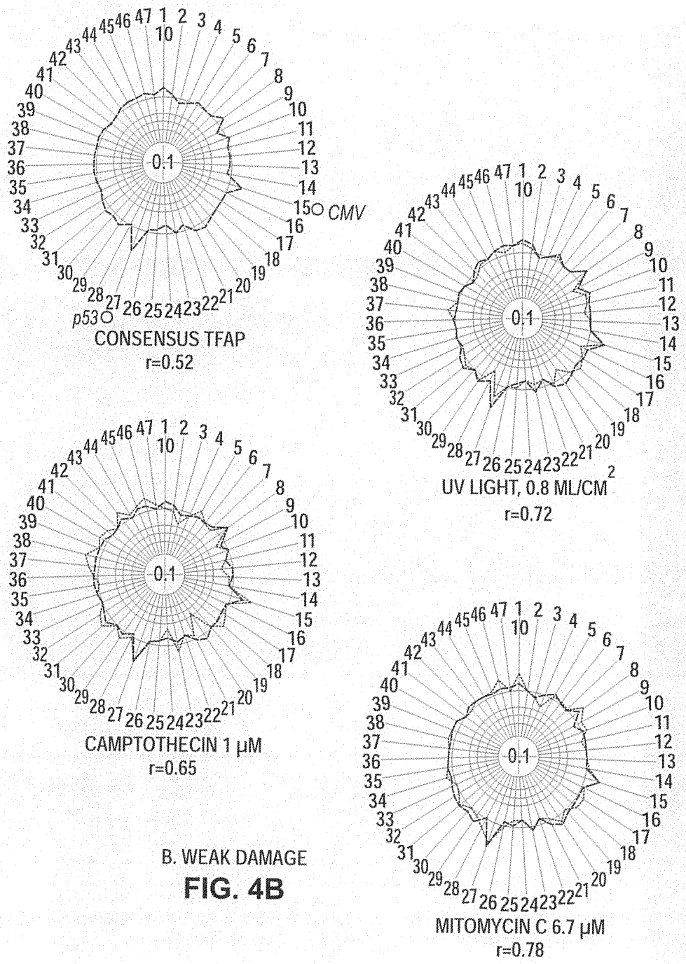

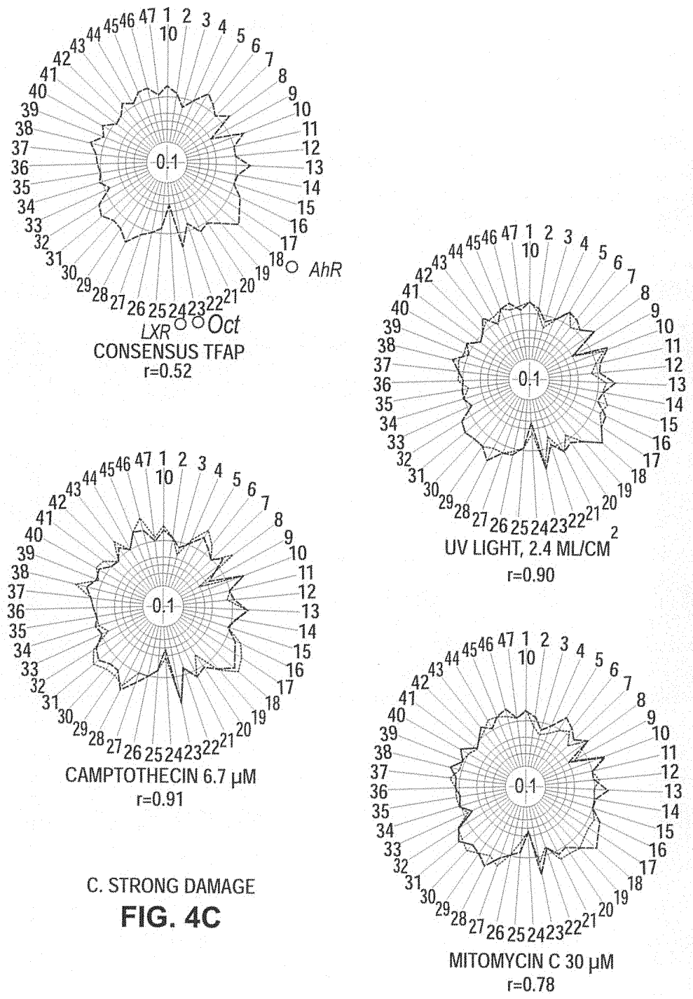

The Invariant TFAPs of DNA-Damaging Agents. To obtain TFAP signatures of DNA damage, HepG2 cells were irradiated by UVC/UVB light or treated with chemicals with different mechanisms of action (MOAs), including topoisomerase I inhibitor camptothecin, topoisomerase II poison auramine O, DNA crosslinking chemicals oxaliplatin, cisplatin, and mitomycin C; and antimetabolites 5-fluorouracil and 5-fluorodeoxyuridine. Cluster analysis at r=0.7 revealed two distinct TFAP clusters with a low similarity consensus signatures (r=0.52) (FIG. 4). One cluster had the TFAPs for a low-dose UV irradiation and for the chemicals at low concentrations (“a weak DNA damage cluster”) (FIG. 4 in Part A). The characteristic features of the consensus TFAP were an activation of AP-1, CMV, and p53 RTUs. The other cluster contained the TFAPs for higher UV doses and for high concentrations of chemicals (“a strong DNA damage”). The consensus TFAP showed multiple responses, including activation of AhR, TCF/b-catenin, ISRE, and downregulation of LXR (FIG. 4, in Part C). Representative TFAPs of the DNA damaging agents are also shown by the heatmap of FIG. 12. Some of these TF responses to DNA damage (e.g., p53, AP-1, and AhR) are well known. Therefore, the UV irradiation and DNA damaging agents produced two different TFAPs at low and high doses. Interestingly, 5-fluorouracil (5-FU) and floxuridine elicited only the weak damage TFAP, while auramine elicited only the strong damage TFAP at all tested concentrations (FIG. 4).

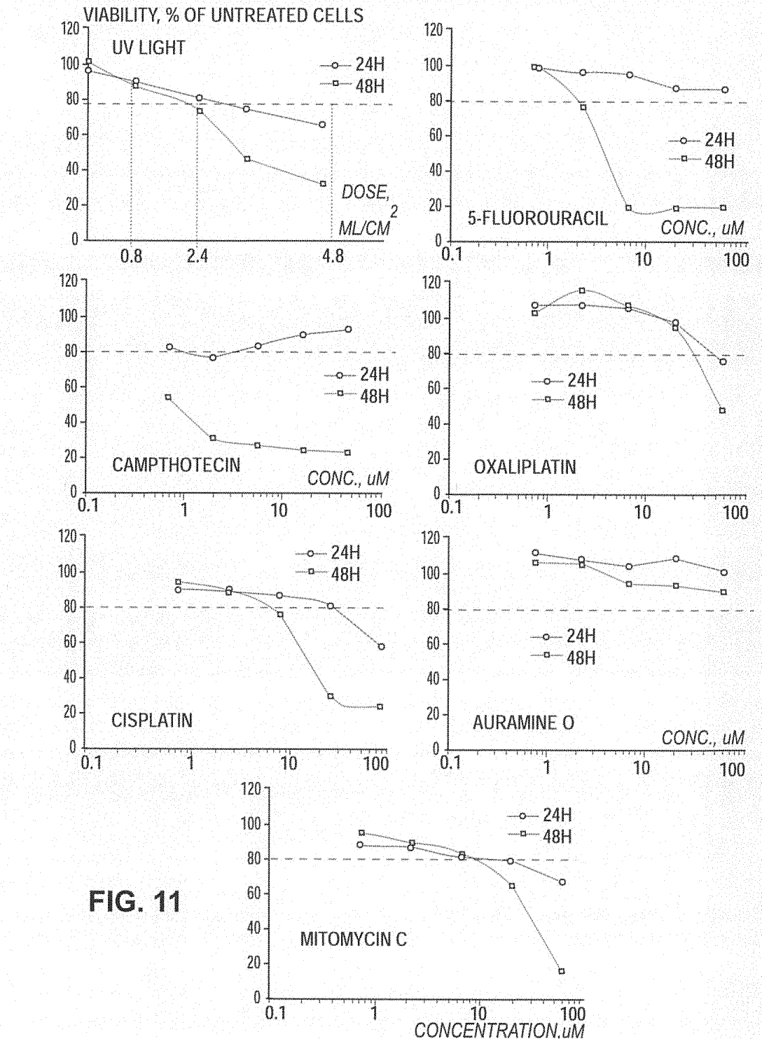

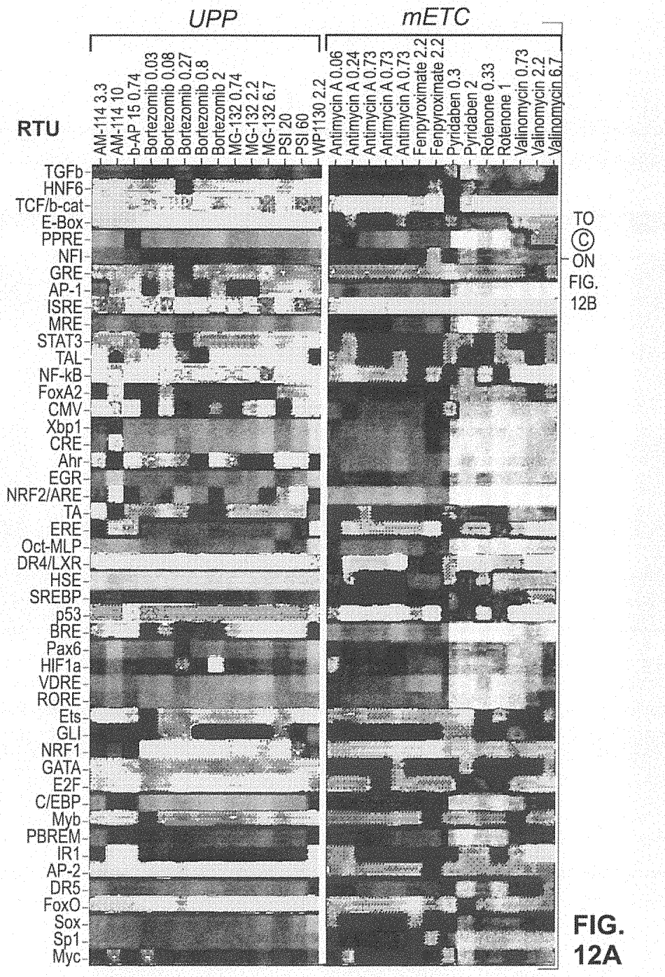

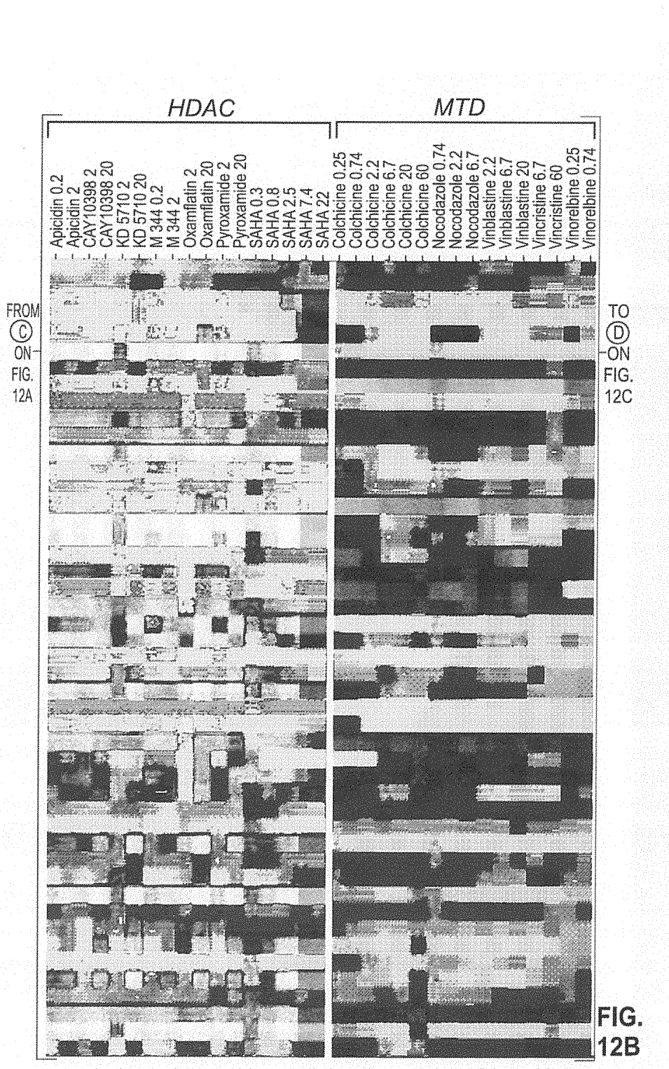

The DNA damaging agents had disparate effects on cell viability; at the end of incubation (24 hours), only some of those (the highest dose of UV and cisplatin) had significantly reduced cell numbers, indicating that the cytotoxicity cannot account for the common TF responses (FIG. 11). The fact that DNA damaging agents produced two distinct TFAPs is consistent with known bi-phasic phenotypic response to DNA damage, when a low-level, repairable damage causes transient arrest of DNA replication, whereas more extensive damage induces permanent arrest of replication.

In summary, the assessment of specific perturbagens of various cell systems and biological pathways showed that each class of perturbagens produced distinct TFAPs. Moreover, perturbagens of the same pathway produced an invariant TFAP, regardless of where and how they interfered.

The Invariant TFAP Signatures Enable Identification of Compounds with Specified Bioactivity.

The finding of the invariant TFAPs demonstrated that these signatures could be used to identify chemicals with specific bioactivities among uncharacterized compounds. To validate such finding, a dataset of TFAP signatures for the environmental chemicals evaluated by Attagene, Inc. under the U.S. Environmental Protection Agency ToxCast project was queried. These chemicals were screened at multiple concentrations using the FACTORIAL™ assay in HepG2 cells. The ToxCast chemicals were also independently evaluated by other groups using different assays. A Tox21 group screened for mitochondria inhibitors using a mitochondria membrane potential (MMP) assay, also in HepG2 cells. The MMP assay data can be found in the EPA Actor database. Using these data, the assessments of mitochondria perturbagens were compared using the invariant mETC TFAP and the functional assay.

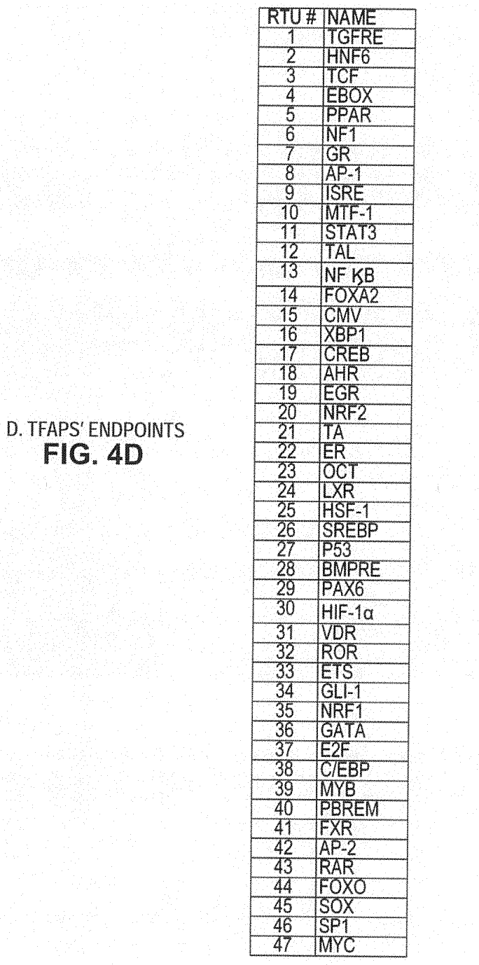

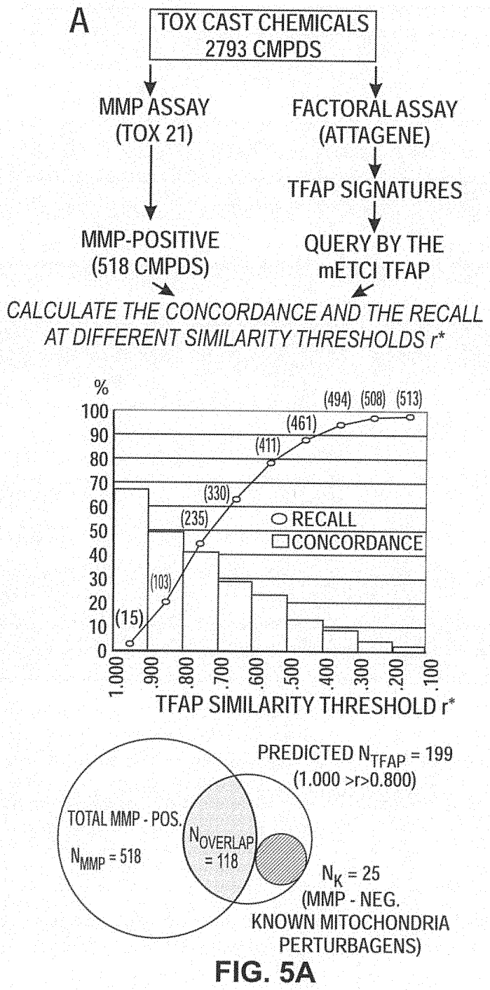

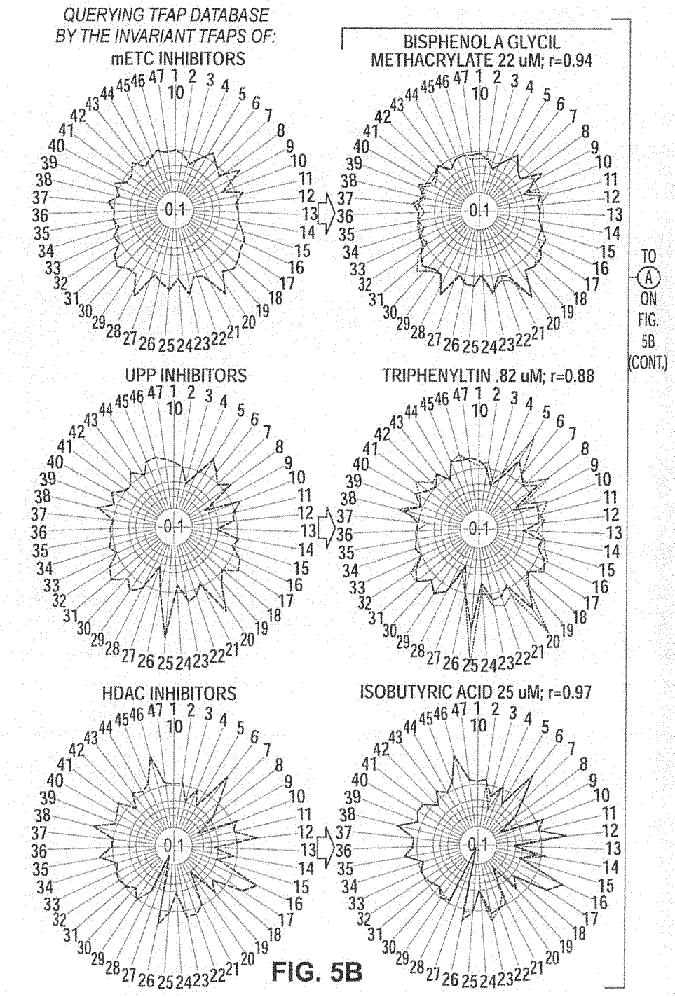

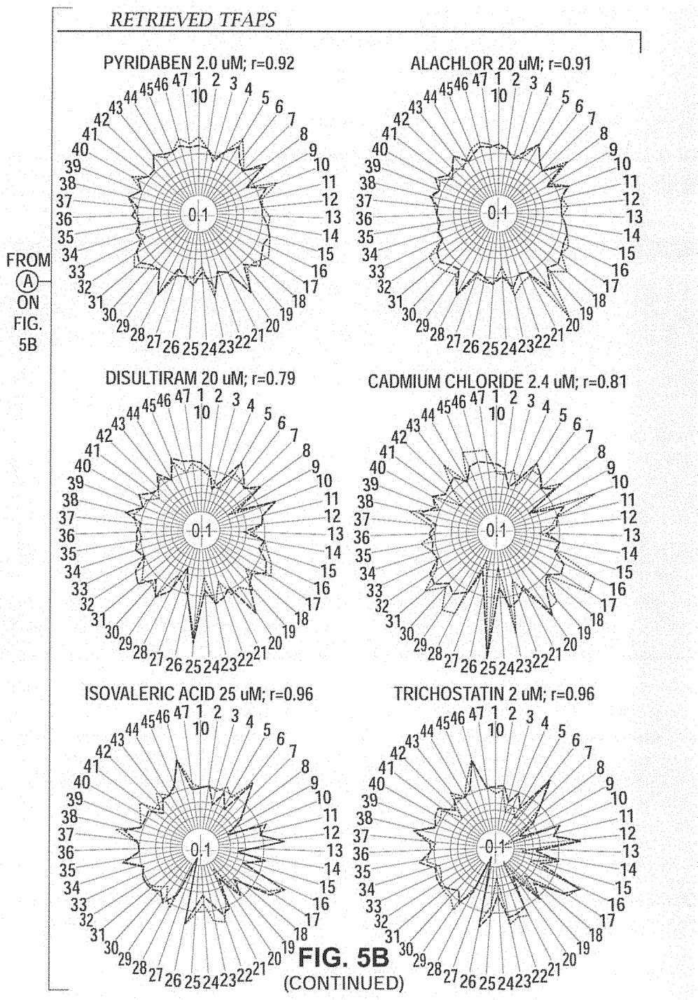

The Actor DB contains data for 2,793 ToxCast chemicals that were evaluated both by the MMP and FACTORIAL™ assays. Of those, the MMP assay scored 518 compounds as mitochondria inhibitors (FIG. 5 in Part A). Querying signatures of these 2,793 chemicals by the invariant mETC TFAP retrieved a large number of chemicals. For some chemicals, multiple TFAPs were retrieved at different concentrations. To avoid redundancy, each retrieved chemical was counted once.

The concordance of the assays' data was calculated as the percent of MMP-positive chemicals among the retrieved chemicals. The concordance rate correlated with the TFAP similarity threshold. For example, ˜69% of retrieved chemicals with the similarity of r≥0.900 were MMP-positive. The chemicals with the similarity values within 0.800≥r≥0.700 had the concordance rate of ˜42% (FIG. 5 in Part A, central panel). Representative retrieved signatures are shown by FIG. 5 in Part B (upper panel).

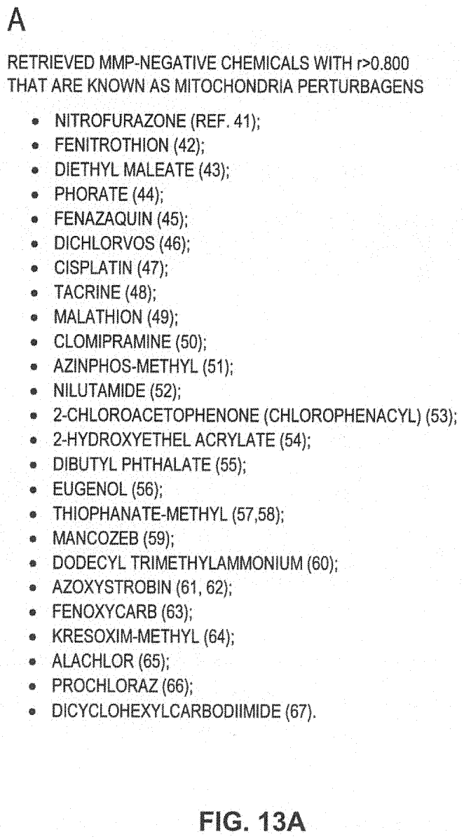

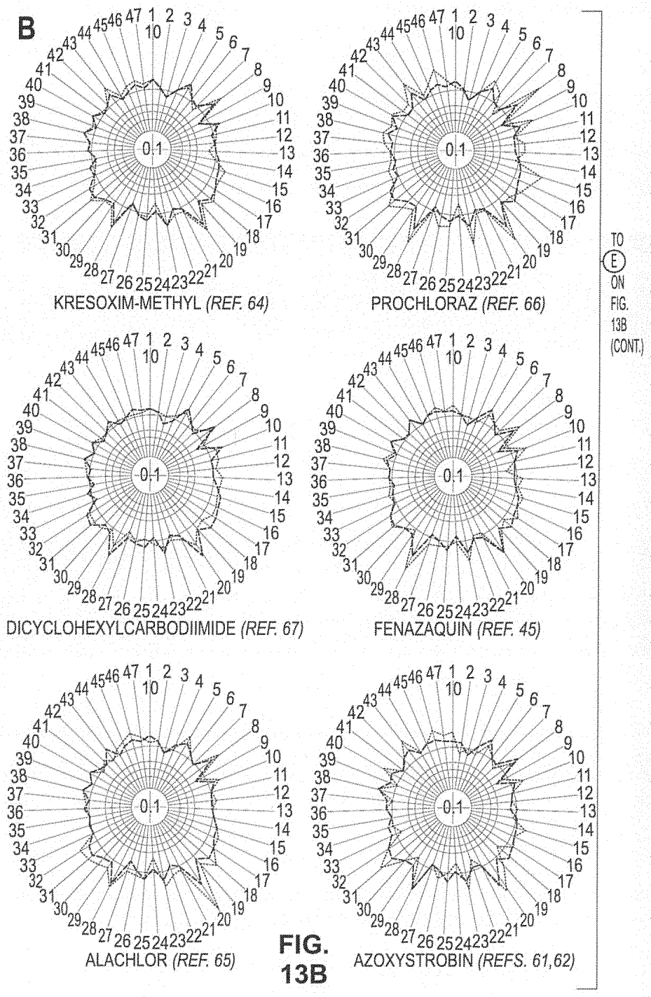

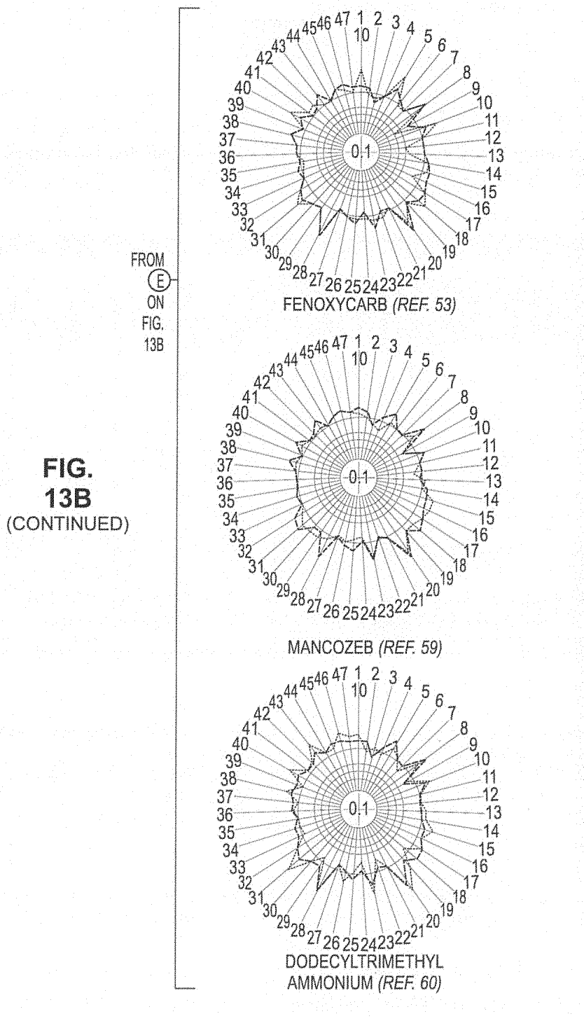

A large fraction of MMP-negatives was found among the retrieved chemicals, and the literature was searched for chemicals with high similarity values. With a similarity threshold at 0.800 199 chemicals were retrieved. Of those, 118 were MMP-positive and 81 were negative (a concordance of 59%) (FIG. 5 in Part A and Table S1). A PubMed search showed that at least 25 of those 81 MMP-negative chemicals were known mitochondria disruptors, such as azoxystrobin and kresoxim-methyl. The FIG. 13 shows the references and representative TFAP signatures for such MMP-negative chemicals, and the TFAP-based predictions provided much better accuracy. One plausible explanation to this discrepancy between the assays is the difference in the screening conditions: the TFAPs were assessed after a 24-hour incubation with concentrations up to 200 μM, whereas the MMP screening entailed a 1-hour incubation at concentrations below 60 μM.

To estimate the cumulative recall of the MMP-positive chemicals, a calculation was made of the number of all 518 MMP-positive chemicals that were retrieved by the TFAP query. The recall inversely correlated with the similarity threshold. With the threshold of 0.900, only 15 MMP-positive chemicals were retrieved (˜2% recall). The recall had increased to ˜49% with the threshold set at 0.700, and to 98% with the threshold of 0.200 (FIG. 5, in Part A, central panel). Thus, setting a high similarity threshold for the query improved prediction accuracy, whereas lower thresholds facilitated a broader coverage.

It was also found that other invariant TFAP signatures afforded the identification compounds with the specified bioactivities. For example, querying the Attagene dataset by the invariant signatures for UPP and HDAC perturbagens yielded known inhibitors of these pathways. FIG. 5, in Part B (central panel) shows representative signatures of retrieved UPP inhibitors, including those for an organotin triphenyltin; an antialcoholism drug disulfiram; and cadmium chloride. Akin to that, querying by the HDAC TFAP retrieved known HDAC inhibitors, including short-chain fatty acids (isobutyric and isovaleric acid) and trychostatin (FIG. 5, in Part B, bottom panel). Therefore, the invariant TFAPs afforded the identification of compounds with the specified biological activities.

The TFAPs Enable the Assessment of the Multiple Bioactivities of Polypharmacological Compounds.

The assessments of specific perturbagens of the biological pathways showed that these chemicals produced unchanged TFAPs in a broad concentration range (FIGS. 2-4). However, it was found that many ToxCast chemicals elicited different TFAPs at different concentrations. One such example was glitazones, antidiabetic drugs that target the nuclear receptor PPARγ and modulate gene expression related to lipid storage, cell differentiation, and inflammation. Another common and PPARγ-independent activity of glitazones is at the mitochondria. Glitazones bind to mitochondrial membranes and specifically inhibit the mitochondrial pyruvate carrier and the respiratory function. The mitochondria malfunction by these drugs has been associated with drug liver injury (DILI). Troglitazone produced most frequent hepatotoxic events and was eventually withdrawn, while pioglitazone and rosiglitazone are on the market.

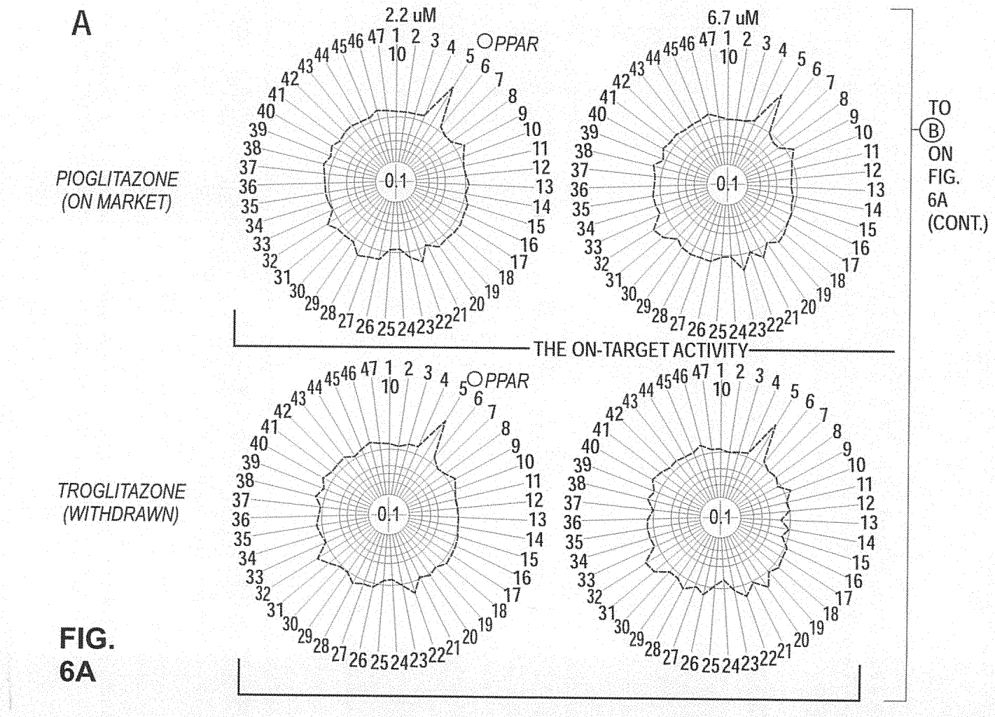

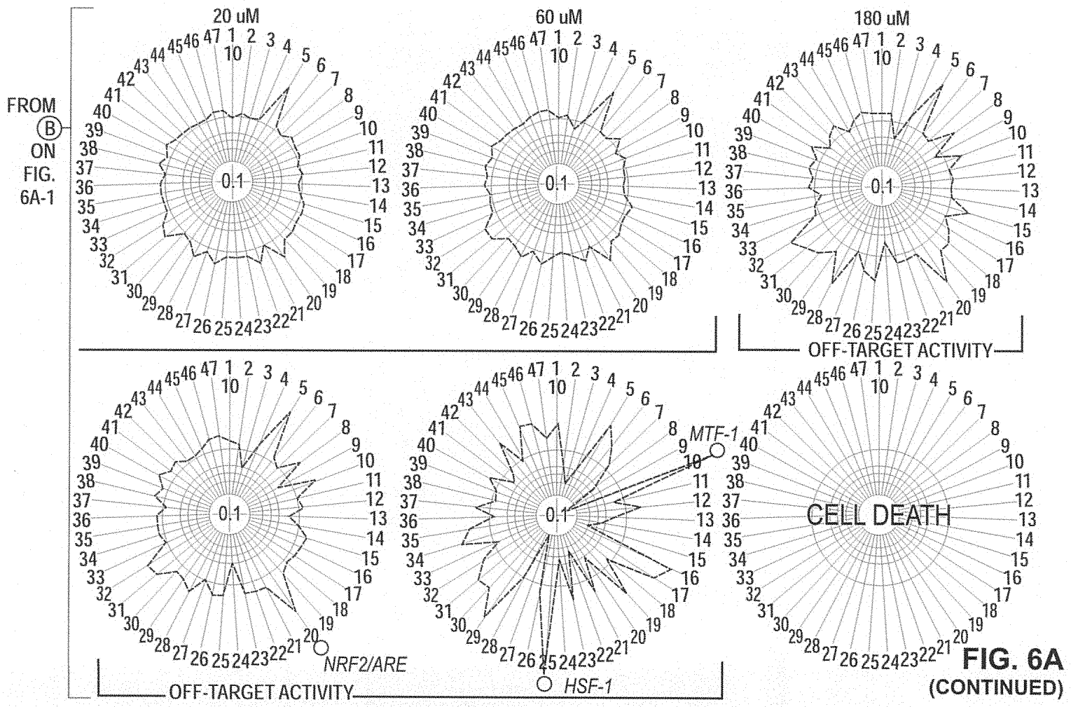

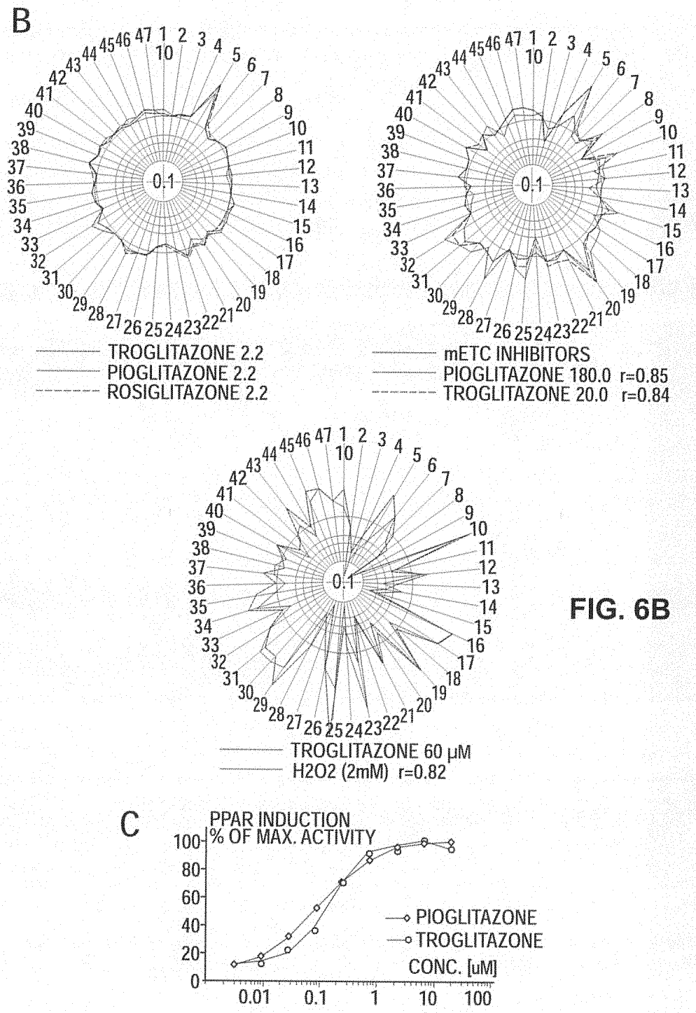

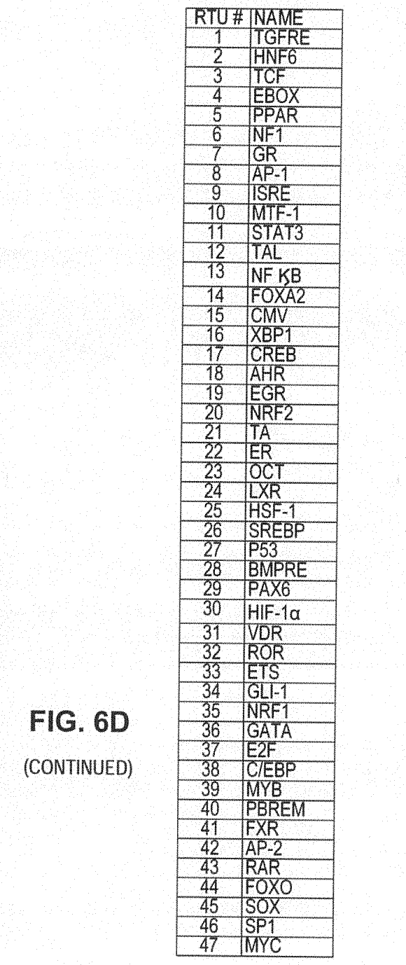

The TFAPs for troglitazone, which is a most-DILI concern drug, and a less-DILI concern pioglitazone, were assessed. The glitazones produced multiple TFAPs at different concentrations (FIG. 6). At low concentrations, both glitazones produced an identical TFAP, which was consistent with their on-target activity (PPAR activation) (FIG. 6 in Part A and FIG. 6, in Part B, left panel). With increased concentrations, the primary TFAPs of glitazones transformed into different, secondary TFAPs, suggesting an influence of off-target drug activities (FIG. 6, in Part A). The signature transformation occurred at different concentrations of troglitazone and pioglitazone (20 μM vs. 180 μM, respectively) (FIG. 6, in Part A). With further increase of concentration (60 uM), the troglitazone TFAP again transformed into a different, tertiary signature (FIG. 6, in Part A). The highest concentrations of troglitazone caused cell death (FIG. 6 in Part A).

The evident similarity of the secondary signatures of troglitazone and pioglitazone suggested a common off-target activity. Querying Attagene TFAP database showed that the secondary TFAPs were identical to the invariant mETC TFAP (r>0.8) (FIG. 6, in Part B, center panel). This indicates an association of the secondary TFAPs with mitochondria malfunction. Querying the TFAP dataset by the TFAP of troglitazone at 60 uM showed its similarity (r>0.8) with the TFAP for hydrogen peroxide (FIG. 6, in Part B, right panel). That suggested that the highest concentrations of troglitazone produced oxidative stress, which is consistent with reports by others.

Therefore, the TFAP signatures afforded the assessment of the primary and off-target activities of glitazones. The signatures at low concentrations reflected the primary drug activity at PPARγ. The reported AC₅₀ values for the PPARγ activation by troglitazone and pioglitazone are of ˜200 nM. These data are consistent with the dose-responses of the PPAR RTU to these drugs (FIG. 6, in Part C). The secondary TFAP indicated mitochondria malfunction and was consistent with the inhibition of the mitochondrial pyruvate carrier by low micromolar concentrations of glitazones.

The data may explain unusually high DILI frequency by troglitazone. The maximum plasma concentrations of troglitazone and pioglitazone in humans are of 6.4 μM and 2.9 μM, respectively. The drugs' TFAPs indicate that these concentrations are below the thresholds for off-target effects (20 μM and 180 μM for troglitazone and pioglitazone, respectively) (FIG. 6, in Part A). However, due to variable genetic and environmental factors, it is possible that drug concentration may exceed the mitochondria malfunction threshold in some individuals. As compared to pioglitazone, therapeutic concentrations of troglitazone are much closer to the off-target threshold, increasing the probability of hepatotoxicity.

The foregoing results demonstrate the utility of TFAP-based assessments for the evaluation of compounds' polypharmacology and the utility of the present methodology in providing a direct and effective approach to bioactivity assessment.

Materials and Methods.

Experimental Design.

Cells and reagents. For the FACTORIAL™ assay in HepG2 cells (ATCC #HB-8065) a HG19 subclone (Attagene, Inc., Research Triangle Park, North Carolina) was selected for an elevated xenobiotic metabolic activity. The MCF7 (ATCC #HTB-22) and HEK293 cells (ATCC #CRL-1573) were from ATCC. Chemical inhibitors were purchased from Cayman Chemical Company (https://www.caymanchem.com) and Selleck Chemicals (www.selleckchem.com). All chemicals were dissolved in DMSO. The ToxCast chemicals were provided by the ToxCast project (U.S. EPA) as 20 mM stock solutions in DMSO. The final concentration of DMSO in cell growth medium did not exceed 0.2%.

UV irradiation. Cells were irradiated using calibrated Spectroline EF-180 UV lamp (Fisher Scientific).

Cell viability was evaluated by the XTT (2,3-Bis-(2-Methoxy-4-Nitro-5-Sulfophenyl)-2H-Tetrazolium-5-Carboxanilide) assay (ATCC) in HG19/HepG2 cells. As a baseline, cells are used that were treated with corresponding dilutions of the vehicle (DMSO). The viability data are an average of two replicates. The viability threshold was set at 80% viability.

The FACTORIAL™ assay was performed. The mix of 47 RTU plasmids was transiently transfected in suspension of assay cells using TransIT-LT1 reagent (Mirus). The transfected cells were plated into 12-well plates. Twenty-four hours later, cells were rinsed and incubated for another 24 h with the evaluated compounds in a DMEM growth medium supplemented with 1% charcoal-stripped FBS and antibiotics. Total RNA was isolated and the RTU activity profiles were assessed by consecutive steps of RT-PCR amplification, HpaI digest, and capillary electrophoresis.

TFAP signatures. The profile of changes of the transcriptional activity of TFs (the TFAP signature) was calculated by dividing the RTU activity values in compound-treated cells by those in vehicle-treated cells. TFAP signatures were plotted as radial graphs comprising 47 axes showing the fold-changes of corresponding RTUs on a logarithmic scale. The value of 1.0 indicated no effects on the TF activity.

Statistical Analysis.

Assessing the similarity of TFAP signatures. A TFAP signature can be viewed as a vector x in a 47-mer space with coordinates x, that are log-transformed fold-induction TF values (log ΔTFᵢ). The length of the vector is calculated as |x|=\sqrt{Σᵢ₌₀⁴⁷(log ΔTFᵢ)²}. The pairwise similarity of TFAP signatures is calculated as Pearson correlation coefficient r (5) that can vary in the range from −1.0 to 1.0.

Calculating the consensus TFAPs for multiple perturbagens. The average linkage method was modified to develop an algorithm for a recurrent agglomerative hierarchical clustering. We start with N clusters {Cⱼ}, j=1 to N, each containing a single TFAP, to find the clusters {Cₖ} and {Cₘ} with the highest similarity r, These clusters are merged into a {Cₖₘ} cluster. The coordinates of the {Cₖₘ} cluster are calculated as an average of xₖ and xₘ vectors, taken with the weights equal to the size of clusters Nₖ and Nₘ, normalized to their lengths |xₖ| and |xₘ|, and multiplied by the average length of these signatures, as following:

$${x}_{k m} = \frac{\vert {x}_{k} \vert \cdot {N}_{k} + \vert {x}_{m} \vert \cdot {N}_{m}}{{( {N}_{k} + {N}_{m} )}^{2}} \cdot ( \frac{{x}_{k}}{\vert {x}_{k} \vert} \cdot {N}_{k} + \frac{{x}_{m}}{\vert {x}_{m} \vert} \cdot {N}_{m} )$$

The resulting TFAP signature is considered as the consensus (average) TFAP for the chemicals within the cluster. The iterative clustering continues until the distance between clusters exceeds a certain similarity threshold r*. In this work, this threshold was set at r*=0.70.

Comparing the TFAP-Based Predictions of Mitochondria Perturbagens with the Functional MMP Assay Data.

The dataset of TFAPs for ToxCast chemicals was queried using the consensus TFAP for mETC inhibitors (FIG. 2, in Part A) and the numbers of retrieved chemicals with the similarity values r within certain intervals (r*₁≥r≥r*₂) were counted. The concordance with the MMP assay was calculated as the percent of MMP-positive chemicals among the retrieved chemicals. To calculate the cumulative recall of the MMP-positive chemicals, we calculated the percent of the 518 MMP-positive chemicals among the retrieved chemicals at different similarity thresholds.

The above-referenced drawing figures are more fully described below.

FIG. 1 illustrates the FACTORIAL™ assay as enabling profiling TF responses to a chemical. Part A shows the flowchart of the FACTORIAL™ assay. The assay cells were transiently transfected with a mix of 47 TF-specific reporter transcription units (RTU). The RTUs contained a restriction tag (the HpaI site) placed at different positions within the reporter sequences. Total RNA was amplified by RT-PCR, using a common pair of primers. The PCR products were labeled with a fluorescent label, digested by the HpaI enzyme, and resolved by capillary electrophoresis (CE). The CE fluorescence profile reflected the TFs activity. The differential TF activity profile (TFAP) for a chemical shows changes of TF activity in the chemical-vs. vehicle-treated cells. By definition, the basal TFAP (in vehicle-treated cells) is a circle with R=1.0. Part B shows the TF endpoints of the FACTORIAL™ assay.

FIG. 2 shows invariant TFAP signatures for inhibitors of the mitochondria function and proteasomal degradation. The assay cells (HepG2) were incubated for 24 h with inhibitors of the mitochondrial electron transport chain (mETC) or the ubiquitin-proteasome degradation pathway (UPP). Each TFAP signature is an average data of three independent FACTORIAL assays. The consensus TFAPs of mETC and UPP inhibitors were calculated by clustering the TFAPs of individual perturbagens as described in Methods. Part A. Representative TFAP signatures of mETC inhibitors. Part B. Representative TFAP signatures of UPP inhibitors. A and B tables show the similarity values r for the TFAPs of the individual perturbagens vs. the consensus TFAPs, calculated as a Pearson correlation coefficient. The radial graphs show TFAPs of perturbagens overlaying the consensus TFAPs. Note that the TF changes values are plotted in a log scale. Part C. The TFAPs endpoints.

FIG. 3 shows invariant TFAP signatures for HDAC inhibitors and cytoskeleton disruptors. The assay cells (HepG2) were incubated with histone deacetylase (HDAC) inhibitors or microtubule polymerization disruptors (MTD) for 24 hours. Each TFAP is an average data of three independent FACTORIAL assays. The consensus TFAPs were calculated by clustering those of individual perturbagens as described in Methods. Part A. Representative TFAP signatures of HDAC inhibitors. Part B. Representative TFAP signatures of MTDs. A and B tables show the similarity values r for the TFAPs of perturbagens at indicated concentrations vs. the consensus TFAPs, calculated as a Pearson correlation coefficient r. The radial graphs show TFAPs of perturbagens overlaying the consensus TFAPs. Part C. The TFAPs endpoints.

FIG. 4 shows that low and high doses of DNA damaging agents produce distinct TFAP signatures. The assay cells (HepG2) were irradiated by a UV source or treated with indicated chemicals and harvested at 24 hours after the treatments. Each TFAP is an average data of three independent FACTORIAL™ assays. The clustering of TFAPs revealed two clusters for the treatments inducing a weak and strong DNA damage. Part A. The table shows the similarity values r for the TFAPs of perturbagens at indicated concentrations vs. the consensus clusters' TFAPs, calculated as a Pearson correlation coefficient. Parts B, C. The radial graphs and show representative TFAPs of perturbagens overlaying the consensus TFAPs. Part D. TFAP endpoints.

FIG. 5 shows that invariant TFAP signatures afford the identification of compounds with specified bioactivities. Part A. Comparing the TFAP-based prediction of mitochondria inhibitors with the data by a functional assay. Upper panel: Total of 2,793 ToxCast chemicals were assessed by the mitochondria membrane potential (MMP) and the FACTORIAL assays. 518 chemicals were scored as mitochondria inhibitors by the MMP assay. The dataset of TFAP signatures was queried by the consensus mETC TFAP. Central panel shows the recall of MMP-positive chemicals (circles) and the concordance rates (bars) for the two assays. The concordance rate is the fraction of MMP-positives among the retrieved chemicals that had TFAP similarity values r within the indicated intervals (r*₁≥r≥r*₂). The cumulative recall curve shows the percent of 518 MMP-positives that were retrieved at different thresholds (r≥r*). Bottom panel: The scaled Venn diagram illustrates the relationship between the two assays. The left area represents 518 MMP-positive chemicals. The right area represents 199 chemicals with the TFAP similarity values r≥0.800. (For the list of retrieved chemicals, see Table S1). The intersection area represents 118 retrieved chemicals that were MMP-positive (the concordance of 118/199˜59%; the recall of 118/518˜23%). The striped area represents 25 known by the literature mETC inhibitors that were scored negative by the MMP assay but positive by the FACTORIAL assay (see also Fig. S7). Part B. Querying the TFAP dataset by the consensus TFAPs of mETC, UPP and HDAC inhibitors had retrieved compounds with corresponding bioactivities. The graphs show the similarity of the retrieved and consensus signatures.

FIG. 6 illustrates an assessment of the on-target and off-target activities of polypharmacological drugs by TF activity profiling. The TFAPs for the glitazones in HepG2 cells (a 24-hour treatment). Each TFAP is an average of three signatures by independent FACTORIAL assays. Representative data of 3 experiments are shown. Part A. The TFAP signature transition with increased concentration indicates an influence of off-target drug activities. Part B. The TFAP signatures enable the identification of the on-target and off-target activities of glitazones. Left panel. The identical TFAPs for low-concentration glitazones reflect the on-target activity (PPAR activation). Center panel. The identical secondary TFAPs for pioglitazone and troglitazone at higher concentrations indicate mitochondria malfunction. Right panel. At the highest concentration (60 uM), the troglitazone TFAP is identical to that of hydrogen peroxide, indicating oxidative stress. Part C. illustrates an assessment of dose-response of the PPAR RTU to glitazones to determine the AC50 values for the on-target activity. Each data point is an average of 3 independent measurements.

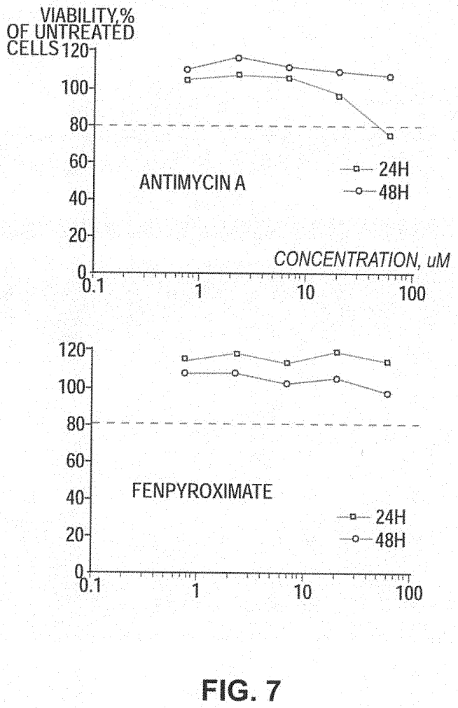

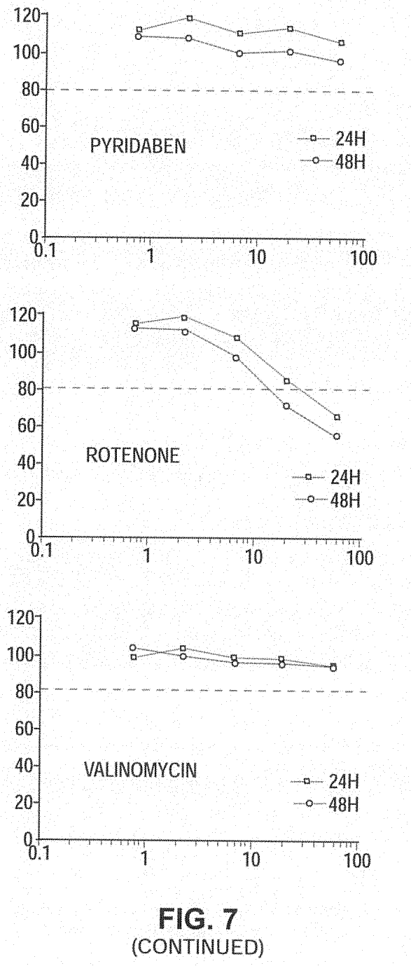

FIG. 7 shows the effect of mETC inhibitors on the viability of assay cells (HepG2).

FIG. 8 shows the effect of UPP inhibitors on the viability of assay cells and the common TFAP signatures for UPP inhibitors in HEK293 and MCF-7 cells.

FIG. 9 shows common TFAP signatures for HDAC inhibitors in HEK293 and MCF-7 cells. and the effect of HDAC inhibitors on the viability of assay cells.

FIG. 10 shows the effect of microtubule polymerization disruptors (MTDs) on the viability of assay cells.

FIG. 11 shows the effect of microtubule DNA damaging agents on the viability of assay cells.

FIG. 12 depicts an alternative presentation of TF responses to perturbagens as a heatmap.

FIG. 13 shows the list of known mitochondria disruptors with a high (r>0.800) TFAP similarity to the mETC TFAP that were scored negative by the MMP assay.

TABLE S1
ToxCast chemicals with a high (r ≥ 0.800) similarity to the invariant mETC TFAP.

| | | | Pearson | | |
|---|---|---|---|---|---|
| | | | correlation | | MMP |
| | | Conc. | with the mETC | | assay |
| # | Chemical | [uM] | TFAP, r | CASRN | hitCall |
| 1 | Bisphenol A glycidyl methacrylate | 22.2 | 0.939 | 1565-94-2 | 1 |
| 2 | Triclosan | 22.2 | 0.936 | 3380-34-5 | 1 |
| 3 | Swep | 200.0 | 0.927 | 1918-18-9 | 1 |
| 4 | Rotenone | 2.0 | 0.917 | 83-79-4 | 1 |
| 5 | Pyridaben | 2.0 | 0.916 | 96489-71-3 | 1 |
| 6 | Fluorosalan | 7.4 | 0.913 | 4776-06-1 | 1 |
| 7 | Prochloraz | 20.0 | 0.912 | 67747-09-5 | 0* |
| 8 | Fenitrothion | 100.0 | 0.912 | 122-14-5 | 0* |
| 9 | Ioxynil | 200.0 | 0.909 | 1689-83-4 | 1 |
| 10 | Dinoseb | 66.7 | 0.908 | 88-85-7 | 1 |
| 11 | Dazomet | 100.0 | 0.907 | 533-74-4 | 0 |
| 12 | Anilazine | 100.0 | 0.906 | 101-05-3 | 0 |
| 13 | Alachlor | 20 uM | 0.906 | 15972-60-8 | 0* |
| 14 | Ipconazole | 22.2 | 0.906 | 125225-28-7 | 1 |
| 15 | Hydroxyflutamide | 200.0 | 0.904 | 52806-53-8 | 1 |
| 16 | Fenamidone | 100.0 | 0.902 | 161326-34-7 | 1 |
| 17 | Mifepristone | 66.7 | 0.902 | 84371-65-3 | 1 |
| 18 | Fipronil | 20 uM | 0.902 | 120068-37-3 | 1 |
| 19 | Besonprodil | 200.0 | 0.902 | 253450-09-8 | 0 |
| 20 | Triclocarban | 66.7 | 0.901 | 101-20-2 | 1 |
| 21 | Triethyltin bromide | 7.4 | 0.901 | 2767-54-6 | 1 |
| 22 | 1-Octen-3-ol | 200.0 | 0.900 | 3391-86-4 | 0 |
| 23 | Norflurazon | 33.0 | 0.898 | 27314-13-2 | 0 |
| 24 | Dinocap | 7.4 | 0.897 | 39300-45-3 | 1 |
| 25 | Flutamide | 66.7 | 0.896 | 13311-84-7 | 1 |
| 26 | N-Isopropyl-N′-phenyl-p- | 22.2 | 0.896 | 101-72-4 | 0 |
| | phenylenediamine | | | | |
| 27 | Azoxystrobin | 66.7 | 0.894 | 131860-33-8 | 0* |
| 28 | Methenamine | 2.5 | 0.893 | 100-97-0 | 0 |
| 29 | Z-Tetrachlorvinphos | 66.7 | 0.892 | 22248-79-9 | 1 |
| 30 | Diethyl maleate | 200.0 | 0.892 | 141-05-9 | 0* |
| 31 | Bifenazate | 100.0 | 0.891 | 149877-41-8 | 1 |
| 32 | Trifloxystrobin | 20.0 | 0.891 | 141517-21-7 | 1 |
| 33 | Metconazole | 66.7 | 0.890 | 125116-23-6 | 1 |
| 34 | Nilutamide | 200.0 | 0.889 | 63612-50-0 | 0* |
| 35 | Indoxacarb | 10.9 | 0.887 | 173584-44-6 | 1 |
| 36 | Kresoxim-methyl | 22.2 | 0.886 | 143390-89-0 | 0* |
| 37 | Picoxystrobin | 22.2 | 0.885 | 117428-22-5 | 1 |
| 38 | Celecoxib | 20.0 | 0.883 | 169590-42-5 | 1 |
| 39 | Fenoxycarb | 20.0 | 0.882 | 72490-01-8 | 0* |
| 40 | Methyl trans-styryl ketone | 66.7 | 0.882 | 1896-62-4 | 0 |
| 41 | 1,1-Dimethoxy-3,7-dimethylocta- | 200.0 | 0.881 | 7549-37-3 | 0 |
| | 2,6-diene | | | | |
| 42 | Parathion | 100.0 | 0.880 | 56-38-2 | 1 |
| 43 | Troglitazone | 22.0 | 0.879 | 97322-87-7 | 1 |
| 44 | 4,4′-(4-Methylpentane-2,2- | 22.2 | 0.879 | 6807-17-6 | 1 |
| | diyl)diphenol | | | | |
| 45 | Profenofos | 13.2 | 0.877 | 41198-08-7 | 1 |
| 46 | Methyl red | 7.4 | 0.876 | 493-52-7 | 0 |
| 47 | Trichlorfon | 100.0 | 0.875 | 52-68-6 | 0 |
| 48 | 2-tert-Butyl-4-methoxyphenol | 200.0 | 0.875 | 121-00-6 | 1 |
| 49 | Butralin | 48.1 | 0.874 | 33629-47-9 | 0 |
| 50 | Oxyfluorfen | 100.0 | 0.872 | 42874-03-3 | 1 |
| 51 | 2,6-Di-tert-butyl-4-nitrophenol | 22.2 | 0.871 | 728-40-5 | 1 |
| 52 | 4-Ethenylphenyl acetate | 200.0 | 0.870 | 2628-16-2 | 0 |
| 53 | Etridiazole | 100.0 | 0.870 | 2593-15-9 | 0 |
| 54 | 1-Chloro-3,3-dimethyl-butan-2- | 66.7 | 0.870 | 13547-70-1 | 0 |
| | one | | | | |
| 55 | Flusilazole | 33.0 | 0.868 | 85509-19-9 | 1 |
| 56 | Rhodamine 6G | 2.5 | 0.868 | 989-38-8 | 1 |
| 57 | Dodecyltrimethylammonium | 7.4 | 0.867 | 112-00-5 | 0* |
| | chloride | | | | |
| 58 | Methyl isothiocyanate | 100.0 | 0.867 | 556-61-6 | 0 |
| 59 | Mancozeb | 22.0 | 0.867 | 8018-01-7 | 0* |
| 60 | Thiophanate-methyl | 100.0 | 0.865 | 23564-05-8 | 0* |
| 61 | Bisphenol AF | 22.2 | 0.865 | 1478-61-1 | 1 |
| 62 | Cyproterone acetate | 66.7 | 0.862 | 427-51-0 | 1 |
| 63 | Oxadiazon | 20.0 | 0.862 | 19666-30-9 | 1 |
| 64 | Bisphenol B | 66.7 | 0.861 | 77-40-7 | 1 |
| 65 | Eugenol | 200.0 | 0.861 | 97-53-0 | 0* |
| 66 | Chlorethoxyfos | 100.0 | 0.860 | 54593-83-8 | 1 |
| 67 | 1-(4-Methoxyphenyl)-1-pentene- | 66.7 | 0.857 | 104-27-8 | 0 |
| | 3-one | | | | |
| 68 | Nitrofurazone | 200.0 | 0.856 | 59-87-0 | 0* |
| 69 | S-Metolachlor | 200.0 | 0.856 | 87392-12-9 | 0 |
| 70 | Propyzamide | 100.0 | 0.856 | 23950-58-5 | 0 |
| 71 | Propargite | 4.8 | 0.856 | 2312-35-8 | 1 |
| 72 | Clorophene | 22.0 | 0.855 | 120-32-1 | 1 |
| 73 | 4-Propylphenol | 66.7 | 0.852 | 645-56-7 | 1 |
| 74 | Dimethomorph | 33.0 | 0.851 | 110488-70-5 | 0 |
| 75 | 2-Cyanoethyl prop-2-enoate | 66.7 | 0.850 | 106-71-8 | 0 |
| 76 | Flufenacet | 100.0 | 0.850 | 142459-58-3 | 0 |
| 77 | Phorate | 200.0 | 0.849 | 298-02-2 | 0* |
| 78 | TNP-470 | 200.0 | 0.848 | 129298-91-5 | 0 |
| 79 | 7-Diethylamino-4- | 200.0 | 0.848 | 91-44-1 | 1 |
| | methylcoumarin | | | | |
| 80 | Bisphenol A | 100.0 | 0.848 | 80-05-7 | 1 |
| 81 | Tetramethrin | 33.0 | 0.847 | 7696-12-0 | 1 |
| 82 | Fluoxastrobin | 3.9 | 0.847 | 361377-29-9 | 1 |
| 83 | Diprop-2-en-1-yl (2Z)-but-2- | 22.2 | 0.846 | 999-21-3 | 0 |
| | enedioate | | | | |
| 84 | Hexythiazox | 33.0 | 0.846 | 78587-05-0 | 1 |
| 85 | Silwet L77 | 7.4 | 0.845 | 27306-78-1 | 1 |
| 86 | 2-Chloro-4-phenylphenol | 66.7 | 0.845 | 92-04-6 | 1 |
| 87 | Chlorfenapyr | 2.5 | 0.844 | 122453-73-0 | 1 |
| 88 | 2,5-Di-tert-butylbenzene-1,4-diol | 66.7 | 0.843 | 88-58-4 | 1 |
| 89 | 2-Chloro-N-phenylacetamide | 22.2 | 0.843 | 587-65-5 | 0 |
| 90 | Binapacryl | 22.2 | 0.842 | 485-31-4 | 1 |
| 91 | 2-Hydroxyethyl acrylate | 200.0 | 0.842 | 818-61-1 | 0* |
| 92 | Nelivaptan | 66.0 | 0.840 | 439687-69-1 | 0 |
| 93 | 3,4,5,6-Tetrachloro-2- | 2.5 | 0.840 | 17824-83-8 | 1 |
| | pyridinecarbonitrile | | | | |
| 94 | Prallethrin | 33.0 | 0.840 | 23031-36-9 | 1 |
| 95 | Dodecyltrimethylammonium | 22.2 | 0.839 | 1119-94-4 | 1 |
| | bromide | | | | |
| 96 | Phosalone | 100.0 | 0.839 | 2310-17-0 | 1 |
| 97 | Bis(2-ethylhexyl) phosphate | 66.7 | 0.838 | 298-07-7 | 1 |
| 98 | Thiazopyr | 20 uM | 0.838 | 117718-60-2 | 0 |
| 99 | Dibutyl phthalate | 100.0 | 0.838 | 84-74-2 | 0* |
| 100 | Benzalkonium chloride | 7.4 | 0.837 | 8001-54-5 | 1 |
| 101 | Famoxadone | 6.2 | 0.837 | 131807-57-3 | 1 |
| 102 | Hexachlorophene | 7.4 | 0.837 | 70-30-4 | 1 |
| 103 | 3,7-Dimethyl-2,6-octadienal | 200.0 | 0.836 | 5392-40-5 | 0 |
| 104 | Dicyclohexyl phthalate | 7.4 | 0.836 | 84-61-7 | 1 |
| 105 | Linuron | 100.0 | 0.836 | 330-55-2 | 1 |
| 106 | Tolazamide | 2.5 | 0.836 | 1156-19-0 | 0 |
| 107 | Resmethrin | 20.0 | 0.835 | 10453-86-8 | 1 |
| 108 | Bisphenol F | 200.0 | 0.835 | 620-92-8 | 1 |
| 109 | 4-Hexylresorcinol | 66.7 | 0.835 | 136-77-6 | 1 |
| 110 | Tetradonium bromide | 22.2 | 0.835 | 1119-97-7 | 0 |
| 111 | Metolachlor | 100.0 | 0.835 | 51218-45-2 | 0 |
| 112 | 2,2′,2″-[Methanetriyltris(benzene- | 7.4 | 0.834 | 66072-38-6 | 0 |
| | 4,1- | | | | |
| | diyloxymethanediyl)]trioxirane | | | | |
| 113 | Flutolanil | 100.0 | 0.834 | 66332-96-5 | 1 |
| 114 | Triadimenol | 17.2 | 0.834 | 55219-65-3 | 0 |
| 115 | Novaluron | 100.0 | 0.833 | 116714-46-6 | 0 |
| 116 | Allethrin | 22.0 | 0.833 | 584-79-2 | 1 |
| 117 | Tiratricol | 200.0 | 0.833 | 51-24-1 | 0 |
| 118 | 2-Chloroacetophenone | 22.2 | 0.832 | 532-27-4 | 0* |
| 119 | Difenoconazole | 6.3 | 0.832 | 119446-68-3 | 1 |
| 120 | Dichlorvos | 100.0 | 0.832 | 62-73-7 | 0* |
| 121 | Bendiocarb | 100.0 | 0.831 | 22781-23-3 | 0 |
| 122 | Ketoconazole | 20.0 | 0.831 | 65277-42-1 | 1 |
| 123 | Cetylpyridinium bromide | 7.4 | 0.831 | 140-72-7 | 1 |
| 124 | 3-(Chloromethyl)pyridine | 200.0 | 0.827 | 6959-48-4 | 0 |
| | hydrochloride | | | | |
| 125 | 2,2′-Methylenebis(ethyl-6-tert- | 22.2 | 0.827 | 88-24-4 | 1 |
| | butylphenol) | | | | |
| 126 | Chlorpyrifos oxon | 100.0 | 0.827 | 5598-15-2 | 1 |
| 127 | Cyazofamid | 23.8 | 0.827 | 120116-88-3 | 1 |
| 128 | Oxycarboxin | 22.2 | 0.827 | 5259-88-1 | 0 |
| 129 | Fenoxaprop-P-ethyl | 200.0 | 0.827 | 71283-80-2 | 0 |
| 130 | Spiroxamine | 33.0 | 0.826 | 118134-30-8 | 0 |
| 131 | Benoxacor | 200.0 | 0.825 | 98730-04-2 | 0 |
| 132 | Tributyltin methacrylate | 0.8 | 0.824 | 2155-70-6 | 1 |
| 133 | Dihexylamine | 200.0 | 0.824 | 143-16-8 | 0 |
| 134 | Ethofumesate | 100.0 | 0.824 | 26225-79-6 | 0 |
| 135 | Dimethyl maleate | 200.0 | 0.824 | 624-48-6 | 0 |
| 136 | Cisplatin | 60.0 | 0.824 | 15663-27-1 | 0* |
| 137 | Azinphos-methyl | 100.0 | 0.823 | 86-50-0 | 0* |
| 138 | 4-Aminoazobenzene | 7.4 | 0.823 | 60-09-3 | 0 |
| 139 | Octrizole | 200.0 | 0.823 | 3147-75-9 | 1 |
| 140 | Dicyclohexylcarbodiimide | 2.5 | 0.823 | 538-75-0 | 0* |
| 141 | 4-Biphenylamine hydrochloride | 7.4 | 0.822 | 2113-61-3 | 0 |
| 142 | Phenazopyridine hydrochloride | 200.0 | 0.822 | 136-40-3 | 1 |
| 143 | Pyraclostrobin | 4.0 | 0.821 | 175013-18-0 | 1 |
| 144 | Elzasonan | 7.4 | 0.821 | 361343-19-3 | 0 |
| 145 | 4-Nonylphenol | 22.2 | 0.821 | 104-40-5 | 1 |
| 146 | Methyl geranate | 200.0 | 0.820 | NOCAS_47125- | 0 |
| 147 | Propanil | 100.0 | 0.820 | 709-98-8 | 1 |
| 148 | 1-Dodecyl-2-pyrrolidinone | 22.2 | 0.820 | 2687-96-9 | 0 |
| 149 | Malathion | 100.0 | 0.819 | 121-75-5 | 0* |
| 150 | Bithionol | 7.4 | 0.819 | 97-18-7 | 1 |
| 151 | Pyriproxyfen | 33.0 | 0.819 | 95737-68-1 | 0 |
| 152 | Cyprodinil | 33.0 | 0.818 | 121552-61-2 | 0 |
| 153 | Diphenylamine | 100.0 | 0.818 | 122-39-4 | 1 |
| 154 | Anise oil | 200.0 | 0.817 | 8007-70-3 | 0 |
| 155 | Daminozide | 100.0 | 0.817 | 1596-84-5 | 0 |
| 156 | 2,4,6-Tribromophenol | 66.7 | 0.817 | 118-79-6 | 1 |
| 157 | 2,5-Bis(2-methylbutan-2- | 7.4 | 0.816 | 79-74-3 | 1 |
| | yl)benzene-1,4-diol | | | | |
| 158 | Spiromesifen | 200.0 | 0.816 | 283594-90-1 | 1 |
| 159 | Kaempferol | 60.0 | 0.816 | 520-18-3 | 1 |
| 160 | Methyl 2-octynoate | 200.0 | 0.815 | 111-12-6 | 0 |
| 161 | Etoxazole | 10.6 | 0.815 | 153233-91-1 | 1 |
| 162 | 2-(2-Ethoxyethoxy)ethyl prop-2- | 200.0 | 0.815 | 7328-17-8 | 0 |
| | enoate | | | | |
| 163 | Loratadine | 20.0 | 0.814 | 79794-75-5 | 1 |
| 164 | 2,4-Hexadienyl isobutyrate | 200.0 | 0.812 | 16491-24-0 | 0 |
| 165 | Diethylstilbestrol | 22.2 | 0.812 | 56-53-1 | 1 |
| 166 | Triadimefon | 100.0 | 0.812 | 43121-43-3 | 0 |
| 167 | Bromoxynil | 100.0 | 0.811 | 1689-84-5 | 1 |
| 168 | Tributyltin chloride | 0.3 | 0.811 | 1461-22-9 | 1 |
| 169 | Rhodamine B | 200.0 | 0.810 | 81-88-9 | 0 |
| 170 | Triphenyl phosphate | 66.7 | 0.810 | 115-86-6 | 1 |
| 171 | Lovastatin | 66.7 | 0.810 | 75330-75-5 | 1 |
| 172 | 4,4′-Dichlorodiphenyl sulfone | 200.0 | 0.810 | 80-07-9 | 1 |
| 173 | Bis[4- | 22.2 | 0.810 | 2095-03-6 | 0 |
| | (glycidyloxy)phenyl]methane | | | | |
| 174 | N,N-Dimethyl-4-nitrosoaniline | 7.4 | 0.810 | 138-89-6 | 0 |
| 175 | Mepronil | 200.0 | 0.810 | 55814-41-0 | 1 |
| 176 | Tacrine | 60.0 | 0.810 | 321-64-2 | 0* |
| 177 | Didecyldimethylammonium | 2.5 | 0.809 | 7173-51-5 | 1 |
| | chloride | | | | |
| 178 | Formetanate hydrochloride | 100.0 | 0.809 | 23422-53-9 | 0 |
| 179 | Pentachlorophenol | 66.7 | 0.808 | 87-86-5 | 1 |
| 180 | Nitrofurantoin | 180.0 | 0.808 | 67-20-9 | 1 |
| 181 | Metsulfuron-methyl | 100.0 | 0.808 | 74223-64-6 | 0 |
| 182 | Eugenyl phenylacetate | 200.0 | 0.807 | 10402-33-2 | 0 |
| 183 | Sodium 2-phenylphenate | 66.7 | 0.807 | 6152-33-6 | 1 |
| | tetrahydrate | | | | |
| 184 | Tris(2,3-dibromopropyl) | 22.2 | 0.806 | 126-72-7 | 1 |
| | phosphate | | | | |
| 185 | Fenazaquin | 0.2 | 0.806 | 120928-09-8 | 0* |
| 186 | Clomipramine hydrochloride | 22.2 | 0.806 | 17321-77-6 | 0* |
| 187 | Pyrimethanil | 100.0 | 0.806 | 53112-28-0 | 0 |
| 188 | Piperonyl butoxide | 100.0 | 0.805 | 51-03-6 | 0 |
| 189 | 1-Chloro-2-(chloromethyl)-3- | 22.2 | 0.804 | 55117-15-2 | 0 |
| | fluorobenzene | | | | |
| 190 | 3-Hydroxyfluorene | 66.7 | 0.804 | 6344-67-8 | 1 |
| 191 | 2-Methoxy-4-methylphenol | 200.0 | 0.803 | 93-51-6 | 0 |
| 192 | Forchlorfenuron | 100.0 | 0.802 | 68157-60-8 | 1 |
| 193 | 2-(Chloromethyl)pyridine | 200.0 | 0.801 | 6959-47-3 | 0 |
| | hydrochloride | | | | |
| 194 | Benfluralin | 100.0 | 0.801 | 1861-40-1 | 0 |
| 195 | Desmedipham | 200.0 | 0.801 | 13684-56-5 | 1 |
| 196 | Benzyldimethyldodecylammonium chloride | 7.4 | 0.801 | 139-07-1 | 1 |
| 197 | Flumioxazin | 100.0 | 0.800 | 103361-09-7 | 0 |
| 198 | 3,3′-Diaminobenzidine | 200.0 | 0.800 | 91-95-2 | 1 |
| 199 | 8-Hydroxyquinoline | 200.0 | 0.800 | 148-24-3 | 0 |

Table S1 shows the listing of retrieved ToxCast chemicals with a high TFAP similarity to the invariant mETC TFAP. The data set of TFAPs for 2793 ToxCast chemicals was queried using the invariant mETC TFAP. The query retrieved 199 chemicals with the TFAP similarity r≥0.800. The TFAPs for many chemicals were retrieved more than once at multiple concentrations. The redundant signatures are not listed in the above table. The HitCall column indicates the matches with the data by the MMP assay (1-yes; 0-no). The asterisks mark the false-negatives of the MMP assay (known from the literature mitochondria disruptors).

In another specific aspect, the present disclosure relates to methodology for assessment of kinase inhibitors, which are compounds that modulate protein or lipid phosphorylation. Kinase inhibitors are an important class of drugs that block certain enzymes involved in the development and progression of diseases such as cancer and inflammatory disorders. Hundreds of kinases exist within the human body, and therefore knowledge of the kinase target of a candidate kinase inhibitor drug is essential for developing successful treatment compositions and strategies. Sometimes clinical trials can fail because candidate drugs bind more than one target. In addition, off-target effects can be beneficial, and candidate drugs can be repurposed for treatment of additional diseases. These considerations mandate a rigorous understanding of the mode of action and effects of such candidate drugs on biological pathways and systems, particularly since the number of targets for which candidate drugs have affinity may widely vary, with some compounds showing exquisite selectivity, and others targeting more than 100 kinases simultaneously, making it difficult to attribute their biological effects to any particular mode of action.

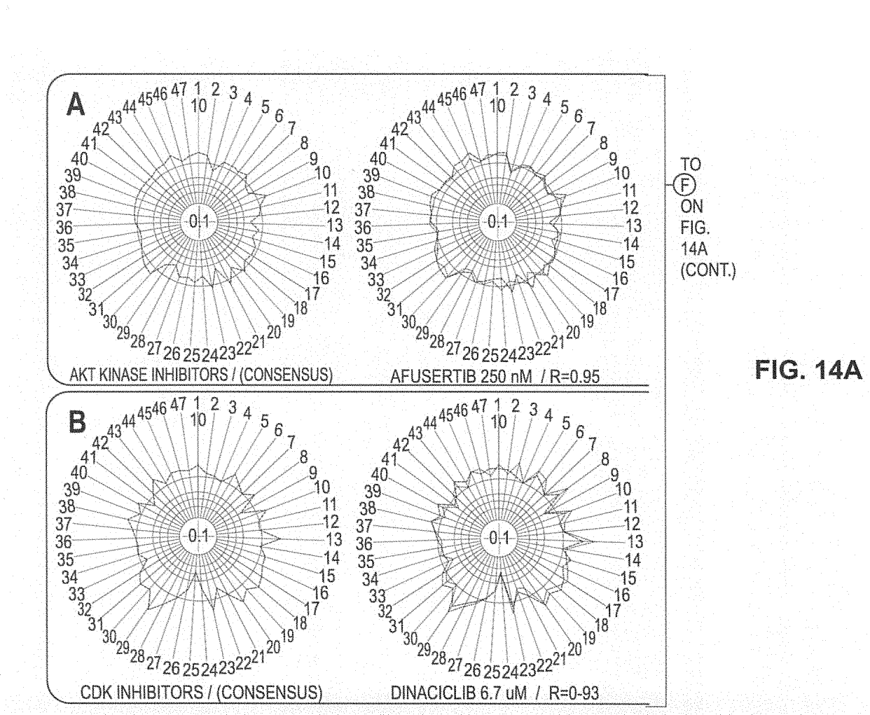

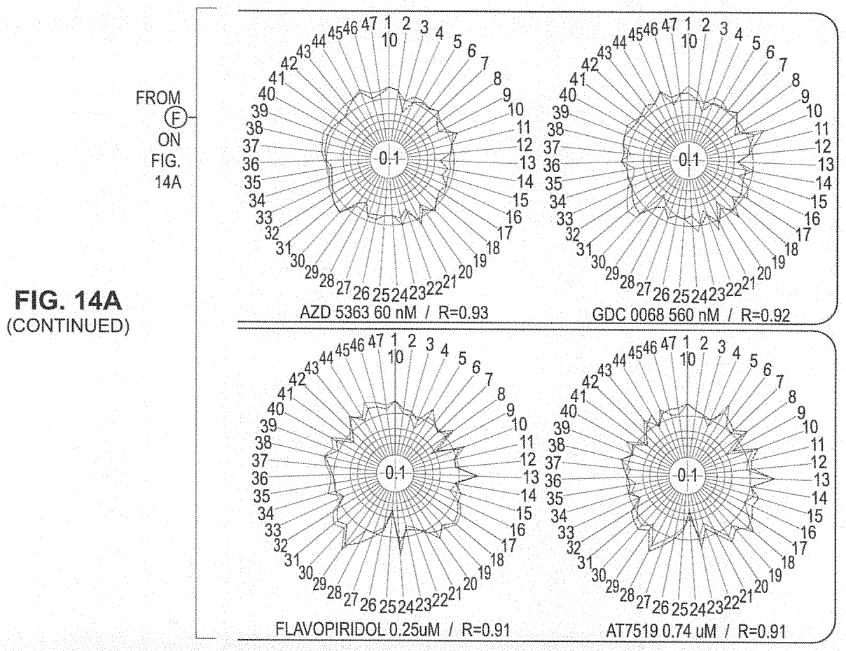

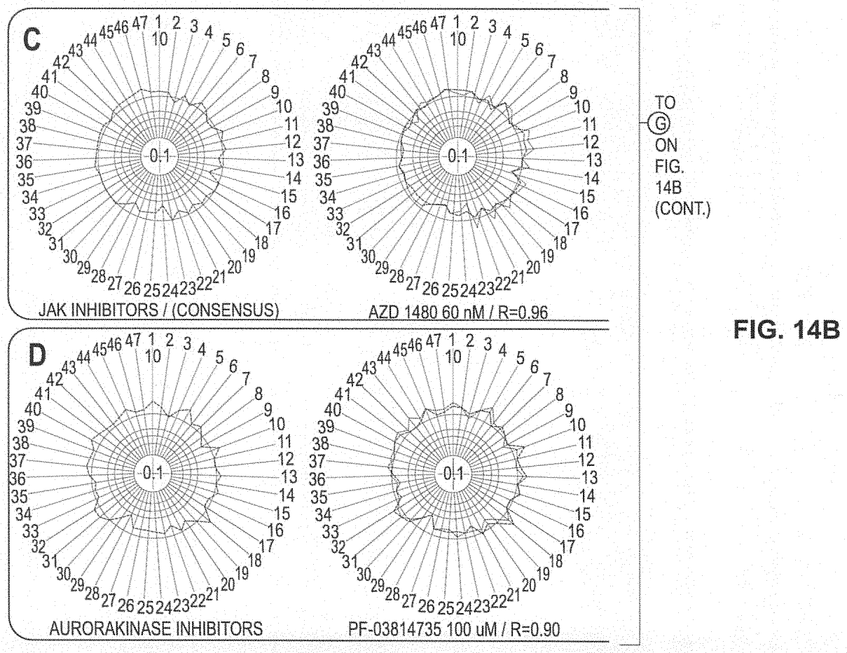

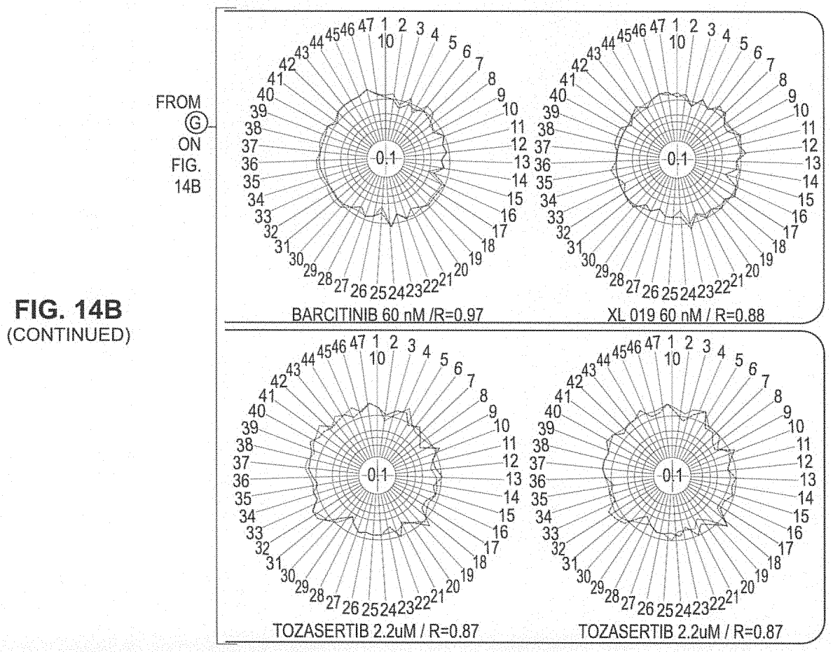

The present disclosure resolves these issues by use of the disclosed methodology to determine the predominant target of a kinase inhibitor. In such methodology, the changes in the activity of transcription factors within kinase inhibitor-treated cells are evaluated with the generation of a TFAP as a simple quantitative signature of seller response. The present inventors have demonstrated that different kinases have distinct TFAP signatures, and that chemically dissimilar inhibitors of the same kinase nonetheless all produce the characteristic invariant TFAP signature. By way of example, FIG. 14 depicts invariant TFAP signatures for four different kinase families: (A) the Akt kinases; (B) cyclin-dependent kinases (CDKs); (C) Jak kinases; and (D) Aurora kinases.

In the generation of the invariant TFAP signatures of FIG. 14, assay cells (HepG2) were treated with indicated kinase inhibitors for 24 hours. The transcription factor activity within cells was determined using the FACTORIAL™ assay, as described in Medvedev et al., Evaluating biological activity of compounds by transcription factor activity profiling, Science Advances, August 2018, the disclosure of which is incorporated herein by reference.

The TFAP signatures are shown in a radial graph format in FIG. 14. Each of 46 axes shows the induced changes in the activity of a particular transcription factor on a log scale. The value of 1.0 indicates no changes in TF activity.

The consensus TFAPs for each kinase class are average signatures of indicated specific kinase inhibitors. The similarity of the individual kinase inhibitors' signatures versus the consensus signatures are shown in FIG. 14.

The transcription factors of the TFAP signatures shown in FIG. 14 are identified in Table 1 below.

TABLE 1
Transcription factors of TFAP Signatures in FIG. 14

| | |
|---|---|
| 1 | TGFb |
| 2 | HNF6 |
| 3 | TCF/b-cat |
| 4 | E-Box |
| 5 | PPRE |
| 6 | NFI |
| 7 | GRE |
| 8 | AP-1 |
| 9 | ISRE |
| 10 | MRE |
| 11 | STAT3 |
| 12 | TAL |
| 13 | NF-kB |
| 14 | FoxA2 |
| 15 | CMV |
| 16 | Xbp1 |
| 17 | CRE |
| 18 | Ahr |
| 19 | EGR |
| 20 | NRF2/ARE |
| 21 | TA |
| 22 | ERE |
| 23 | Oct-MLP |
| 24 | DR4/LX |
| 25 | HSE |
| 26 | SREBP |
| 27 | p53 |
| 28 | BRE |
| 29 | Pax6 |
| 30 | VDRE |
| 31 | RORE |
| 32 | Ets |
| 33 | GLI |
| 34 | NRF1 |
| 35 | GATA |
| 36 | E2F |
| 37 | C/EBP |
| 38 | Myb |
| 39 | PBREM |
| 40 | IR1 |
| 41 | AP-2 |
| 42 | DR5 |
| 43 | FoxO |
| 44 | Sox |
| 45 | Sp1 |
| 46 | Myc |

Protein kinases are 1 of the most promising classes of drug targets for cancer, diabetes, inflammation, and other diseases. Kinase drug discovery efforts are complicated by multiple factors. One is a significant similarity of kinase active sites. Consequently, most PK inhibitors (PKIs) interact with multiple kinases. Another problem is that biological functions of many kinases are unknown and adequate chemical probes for their elucidation are missing. Existing approaches permit profiling PKI activity across the kinome but do not provide clear guidance for prioritization of lead candidates. Furthermore, these approaches do not account for interactions with non-kinase targets constituting the bulk of cellular proteins.

The disclosure therefore contemplates a novel methodology to evaluate biological properties of PKI's that are invisible to existing, target-based approaches. The present approach will aid in identifying differences between PKI series to prioritize optimal leads. Furthermore, it will enable a systematic functional annotation in the context of vertical signaling pathways. The determination of TFAPs provides a robust quantitative signature enabling straightforward identification of perturbed biological processes and systems.

The present inventors have found that inhibiting a kinase by structurally dissimilar PKIs or by kinase-dead cDNAs produce a common TFAP signature, and this invariant signature provides a specific marker for the inhibited kinase. Distinct invariant signatures for multiple kinases have been demonstrated (e.g., Raf, Mek, Erk, mTOR, Akt, CDKs, and Aurora), and it has been found that kinases of the same vertical pathway had similar invariant signatures, thereby identifying a new approach to functional annotation of kinases within the signaling network. A TFAP-based approach for assessment of multiple biological activities (polyphonic) of PKI's is provided by the present disclosure. The TFAP signature of an individual PKI changes with concentration; at some concentrations (the specificity window), it matches the invariant signature for a particular kinase—at other concentrations, the invariant signatures of perturbed non-kinase biological processes (e.g., mitochondrial malfunction, DNA damage, etc.). Thus, TFAP signatures provide definitive and quantitative metrics for assessing multiple bio activities of PKI's.

The FACTORIAL™ assay enables screening of inhibitors for kinase with known biological functions. Using multiple PKI's per kinase, invariant signatures can be identified for kinases. The signatures can be validated as specific markers of inhibited kinase by use of kinase-dad cDNA mutants.

To assess polyphonic of individual PKI's and build a map of vertical kinase pathways for the illuminated kinome, multiple bioactivities can be evaluated by determining specificity windows for target kinases that dominate cell response and perturbed non-kinase biological processes. These data can be assembled into a database of bioactivity profiles for the PKI's, following which the similarity of the invariant signatures for kinases can be analyzed to annotate their places within vertical kinase pathways. Such map can be validated using literature and biochemical assays.

The present disclosure further contemplates invariant signatures for “dark kinases” that can be integrated into the map of the illuminated kinome. Since selective inhibitors for dark kinases may not be available, kinase-dead cDNAs may be employed to obtain invariant signatures for integration of dark kinases into the illuminated kinome map.

The disclosure further contemplates the generation of chemical probes for dark kinases. Using invariant signatures for dark kinases, novel inhibitors can be identified among screen PKI's. Using medicinal chemistry these inhibitor series can be developed to obtain derivatives with extended specificity windows. Selectivity of the obtained chemical probes may be assessed using a proteomics-based kinase profiling technique.

The disclosure therefore enables assessment protein kinases in a direct and effective manner. The human kinome comprises an estimated 518 kinases, and many inhibitors interact with multiple kinases. Resulting polyphonic can compromise drug safety, but in some situations, inhibition of multiple kinases can greatly enhance therapeutic efficiency. In addition, many kinases are poorly characterized regarding their places in cell signaling, target spaces, and biological functions. Accordingly, the roles for many dark kinases in disease and their significance as potential drug targets are largely unknown. A major obstacle is the scarcity of appropriate chemical probes permitting selective inhibition of particular kinases. Another serious issue relates to the fact that PKI' s, like many other drugs, can interact with non-kinase targets, such as bromodomain-containing proteins, cytoskeleton, prostaglandins synthases, the AHR receptor, ferrochelatase, NQO2, and others. As the kinome represents less than 0.1% of all human proteins, the exploration of the role for PKI interactions with non-kinase targets is essential. At present, the impact of these non-kinase interactions on drug safety and efficacy are poorly understood.

In this respect, the methodology of the present disclosure affords a significant advance in the art in relation to conventional technologies for PKI evaluation, and proteomic-based approaches. The limitations of these approaches are addressed and resolved by the methodology of the present disclosure, of evaluating the biological properties of kinase inhibitors by TF activity profiling. The disclosure therefore presents a novel, effect-based PKI evaluation approach, in which the biological properties of a PKI is inferred by assessing responses of the network of cellular signaling pathways. As a readout, the activity of transcription factors that connect signaling pathways with regulated genes are assessed. Since transcription factor transcriptional activity, i.e., its ability to activate or inhibit transcription, is regulated by signaling pathways, the profile of TF activity captures the state of cellular signaling. The invariant TFAPs provide specific markers for perturbed biological processes and cell systems.

The FACTORIAL™ assay permits the assessment of multiple reporter construct in a single will of cells, with unprecedented accuracy and reproducibility, evaluating transcription factor transcriptional activity as opposed to simply TF protein or RNA expression. This is critically important, since TF activity is regulated by different post-translational modifications (phosphorylation, acetylation, methylation, etc.) that do not alter the content of pre-existing TF proteins.

The FACTORIAL™ assay characterizes compound of interest by a TF activity profile (TFAP), a quantitative multi-endpoint signature showing the activation or inhibition of evaluated TFs in compound-treated cells. The standard assay covers 46 TF families (including AP-1, NF-κB, NRF2, HIF-1a, etc.) that govern transcriptional responses to a variety of stress stimuli, cytokines, and growth factors. The advantage of TFAP-based description is that TFAP signatures are quantitative descriptors of cell response, allowing for straightforward comparisons of cell responses to different compounds by simple parametric statistics (e.g., Euclidean distance or Pearson correlation).

The present inventors have found that most inhibitors for a given kinase shared a common TFAP signature, regardless of their structural dissimilarities and mechanisms of binding (allosteric vs. ATP-competitive inhibitors). Moreover, the signature produced by genetic kinase inactivation (i.e., by expressing kinase-dead cDNA) was identical to the common signature of chemical inhibitors, as established by empirical data. Combined, these data provided compelling evidence that inhibition of a particular kinase produces coordinated changes in the activity of signaling pathways, epitomized by a specific TFAP signature. These invariant signatures for inhibited kinases also provide specific markers for the “on-target” activity of kinase inhibitors.

An individual PKI can exhibit multiple signatures at different concentrations. Importantly, at some concentrations, different PKIs of a given kinase exhibited a common TFAP, and the same signature was produced by specific kinase inactivation by genetic tools, establishing that this common PKI signature as an invariant signature and a specific marker for inhibited kinase. This important distinction opens the unique opportunity to use TFAP profiling both to discover selective PKIs and to assess the multiple activities of individual PKIs.

In studies conducted by the present inventors, TFAP signatures for individual PKIs were assessed in a broad range of concentrations. It was found that, in most cases, the PKI TFAP changed with concentration. The TFAPs of a given PKI had a high similarity (r>0.7) to the invariant signature of its intended kinase target only within certain concentration range (“the specificity window”); at other concentrations, distinctively different signatures were observed. Within the specificity window, the on-target PKI activity dominated cell response; outside of this window, other PKI activities (toward off-target kinases or/and non-kinase targets) had also affected cell responses, as reflected by the changed TFAP signature. To delineate the underlying effects for PKI TFAP signatures, datasets were queried, of (a) the invariant signatures for inhibited kinases and (b) invariant TFAPs for perturbagens of non-kinase targets (i.e., mitochondria, proteasome, and HDAC inhibitors; DNA damage, etc.). The purpose was to find the invariant signatures that had the highest similarity to the TFAP of given PKI at given concentration. This way, it was sought to characterize the PKI activities dominating cell response at different PKI concentrations.

It was resultingly found PKIs that had a single specificity window for the intended kinases within a broad concentration range. Therefore, these PKIs may be considered as preferred selective candidate chemical probes for the kinases. There were also PKIs with multiple specificity windows for different kinases. Interestingly, at the highest PKI concentrations, signatures were found that suggested perturbations of particular non-kinase processes, such as mitochondrial malfunction, HDAC inhibition, and oxidative stress.

Accordingly, the TFAP-based approach provides clear quantitative metrics for comprehensive biological assessment of PKIs. Using the TFAP signatures of a PKI, concentration ranges (specificity windows) can be determined, wherein the cell response is dominated by the inhibition of particular kinases. Furthermore, the TFAP-based metrics enable straightforward identification of perturbed nonkinase biological processes and systems in PKI-treated cells. These data indicate that incorporation of TFAP signatures into a lead optimization process provides a greater depth of biological annotation that will add significant value in the prioritization and optimization of lead PM candidates.

In various embodiments of the present disclosure, mining tools such as the Chemotext (chemotext.mml.unc.edu) and the ROBOKOP (robokop.renci.org) may be employed to identify both clinical and adverse outcomes pathways associated with both kinase-selective PKIs and those with polypharmacological mechanisms of action.

In various implementations of the disclosure, the invariant signatures of inhibited kinases may be employed to discover or develop novel PKIs with specified on-target activities. The present inventors have determined that the invariant signatures of perturbagens of biological processes allowed for the identification of novel perturbagens with a specified activity among uncharacterized chemicals. For example, the invariant signatures for inhibited Akt and CDK kinases were employed to to query the Attagene DB database containing >30,000 TFAP entries across a diverse chemical collection, resulting in the retrieval of a substantial number of chemicals with TFAP signatures highly similar to those signatures characteristic of Akt or CDK. Literature searches showed that some of the retrieved chemicals did inhibit these kinases. Therefore, the invariant TFAPs of inhibited kinases can permit the identification of novel PKIs with specified activities among uncharacterized chemicals. The FACTORIAL™ process can also add value to traditional kinase inhibitor optimization efforts by defining the concentration range over which an on-target cellular response is maintained, which is, in essence, a measure of functional selectivity in cells. This approach may be employed to identify and evaluate novel PKIs among approved drugs, environmental chemicals, and other chemical starting points. The identified compounds can provide candidates for drug repurposing and/or can be used as starting points for synthesizing more selective kinase inhibitors. Cheminformatics approaches can be utilized to elucidate possible structural determinants of PKIs that display specific TFAP signatures.

The invariant signatures of inhibited kinases permit functional annotation of kinases in the context of vertical kinase pathways. Analysis of the invariant signatures for inhibited kinases demonstrate that the signatures for kinases of the same pathway (e.g., AKT and mTOR; RAF, MEK, and ERK) have a high similarity. Moreover, kinases that were more proximal had higher similarity of their invariant signatures. Using an agglomerative clustering algorithm, a map of kinase pathways was prepared for the AKT, mTOR, RAF, MEK, ERK, CDKs, and Aurora kinases. This map was fully consistent with the well-established roles for these kinases in vertical signaling pathways. The present disclosure methodology enables the buildout of the map for a substantial part of the human kinome and integrate dark kinases into the context of the illuminated kinases' signaling pathways. Such maps can be employed to elucidate the biological role for the dark kinases and thus aid in their deorphanization.

The present disclosure thus provides a novel, effect-based methodology to evaluate biological properties of kinase-targeting drugs that are invisible to existing, target-based screening approaches. This new systems biology approach enables the identification of differences between PKI series and allow for prioritization of safer leads. Furthermore, this methodology enables development of a systematic functional annotation of human kinases in the context of vertical signaling pathways.

Whereas conventional PKI selectivity profiling techniques characterize PKIs by their binding affinity and effects on kinase activity, the present methodology infers the biological activities of PKIs by profiling responses of cellular signaling pathways. The obtained signatures permit straightforward identification of PKI targets and define the concentration windows where the on-target and off-target activities dominate the cell response, thereby achieving a fundamental advance in the art over such conventional techniques. TFAP signatures are simple quantitative descriptors allowing for direct identification of perturbed processes and systems, in the practice of the methodology of the present disclosure.

To profile TF activity in response to PKIs, the FACTORIAL™ reporter assay is advantageously employed, utilizing transient transfection of test cells with a set of reporter constructs with TF-inducible promoters producing reporter RNAs proportionate to TF activity. By profiling RNA transcripts we assess TF activity profile. To ensure accuracy and reproducibility of the parallel detection, the assay uses an RNA profiling approach, according to which all RTUs have identical reporter sequences. To facilitate detection, the RTU reporter sequences are “tagged” by a unique HpaI restriction site. The reporter RNAs are amplified by RT-PCR, using one pair reporter sequence-specific primers. The PCR products are labeled by a fluorescent label and digested by the HpaI enzyme, producing DNA fragments of predetermined sizes that are separated and detected by capillary electrophoresis (CE). From the CE data, the TF activity profile (TFAP) is calculated. To describe cell response to a compound, a differential TFAP signature showing changes of TF activity is generated, and PKI TFAP signatures as radial graphs with axes showing fold-changes of TF activity in a log format. By definition, the TFAP signature of vehicle-treated cells is a perfect circle with R=1.0.

The present inventors have demonstrated the methodology of the present disclosure with human hepatocarcinoma cells (HepG2) that were treated with PKI's for 24 hours. Pairwise similarity of TFAP signatures were assessed by Pearson correlation coefficients (r). The probability that 2 random 48-end point TFAP signatures have a similarity r>0.7 is less than 10⁻⁷. To compare multiple signatures, the average linkage method has been modified to develop an algorithm for agglomerated hierarchical clustering, in which the algorithm group similar TFAP signatures into clusters with predetermined similarity thresholds and calculates the central (average) signatures for the clusters.

To perform systematic analysis of PKI signatures, the agglomerative clustering algorithm was employed, with collection of signatures for all inhibitors of a given kinase at multiple concentrations to identify clusters of similar signatures.

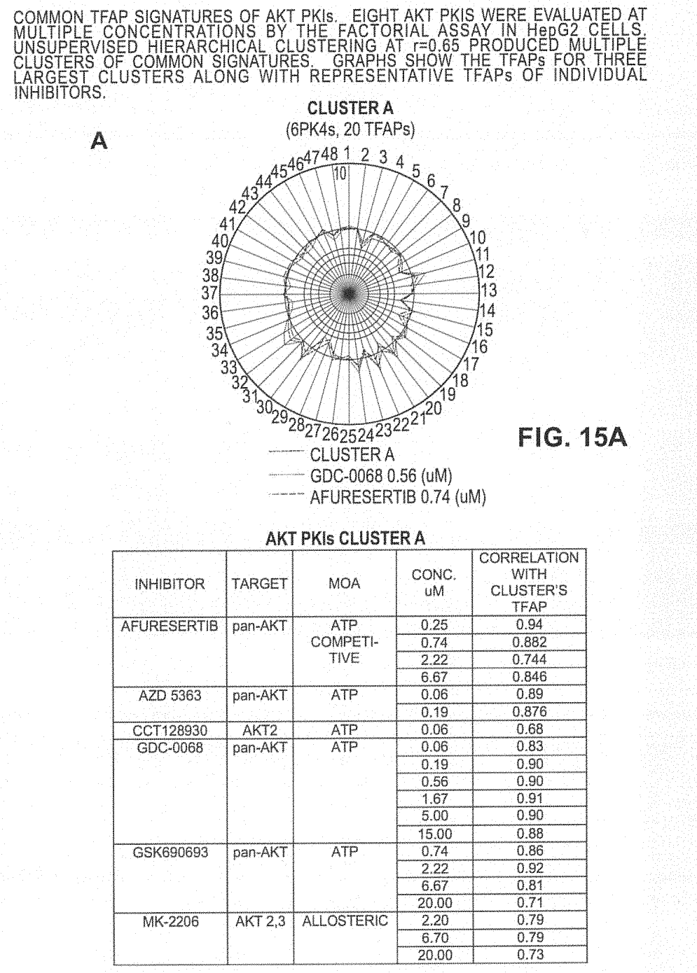

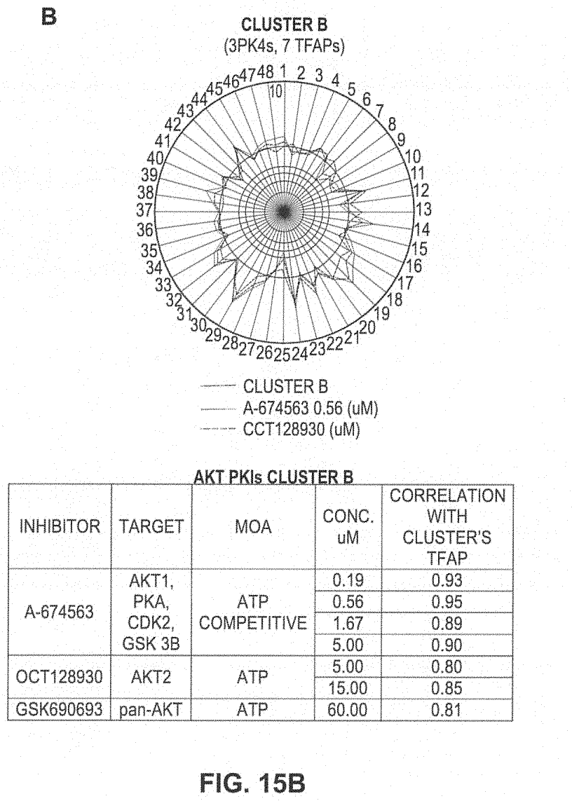

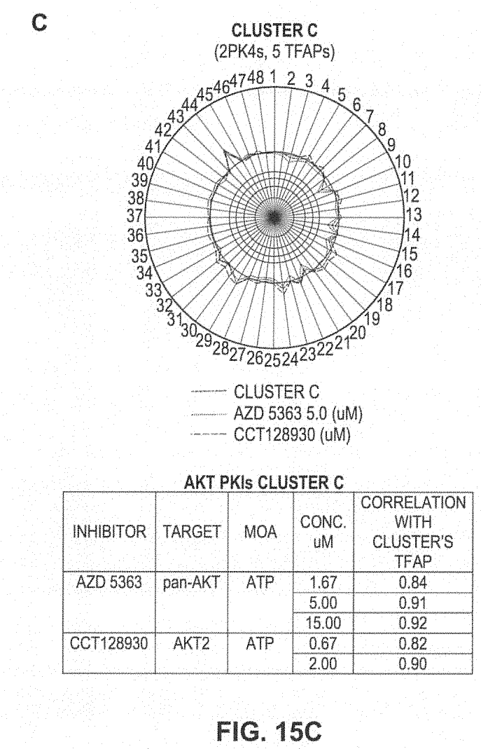

FIG. 15 shows that there was the main cluster A, containing signatures for most (6 out of 8) Akt inhibitors, and minor clusters (B and C), each containing a few signatures.

Importantly, the main cluster contained signatures of structurally diverse inhibitors with different MOAs (allosteric and ATP-competitive). Consequently, this common signature was identified as the invariant PKI signature for Akt kinase. The minor clusters may reflect PKIs' activities toward off-target kinases other than Akt.

Akt was inactivated using ectopic expression of a kinase-dead Akt cDNA mutant (K179A). It was found that the KD mutant TFAP was identical to the consensus signature for the major cluster (A) of Akt PKIs (r=0.85). Thus, the common, the invariant signature for PKI inhibitors provides a specific marker of inhibited kinase.

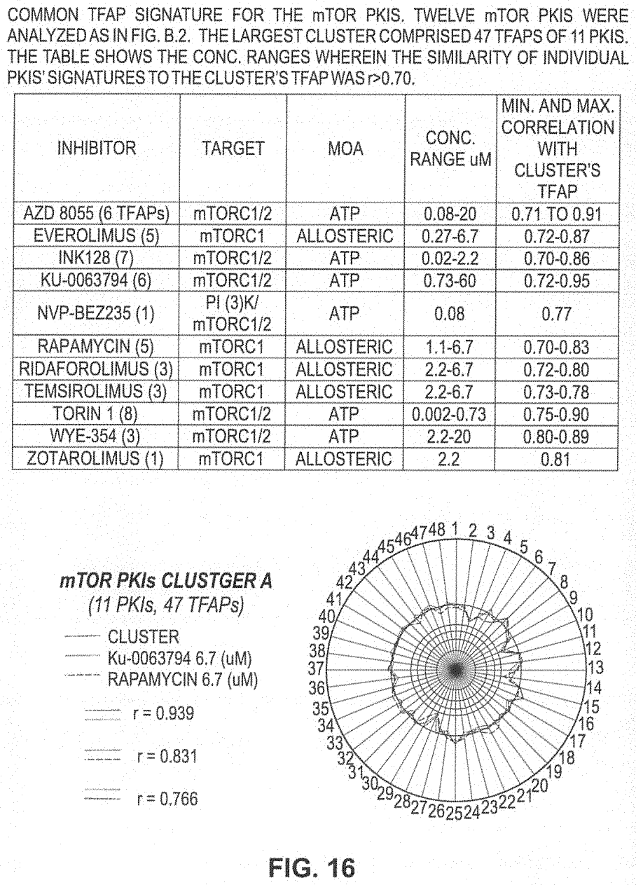

The evaluation of inhibitors of mTOR, CDK, RAF, ERK, MEK, and Aurora showed that each kinase had its own, distinct invariant PKI signature (FIG. 16).

Most individual kinase inhibitors exhibited a variable TFAP signature that changed with PKI concentration. At some concentrations (“specificity window”), this signature coincided with the invariant signature for the target kinase. The variability of individual PKI TFAPs was attributed to the influence of PKI effects on off-target kinases and non-target biological processes. To reveal the underlying effects, we analyzed some PKIs by querying their multiple signatures against the Attagene DB database containing TFAP signatures for compounds with known bioactivities.

CDK2 inhibitor NU6140 elicited three different signatures at different concentrations. From 0.25 to 2.2 uM, its signature was similar to the invariant TFAP of Aurora inhibitors; at 6.7 uM, to the invariant signature for CDK inhibitors; at 20 uM, to the signature for a cadmium salt (r=0.71). In contrast, a selective CDK inhibitor Dinaciclib over three logs of concentrations showed a steady signature that had a high similarity (r>0.85) to the invariant signature for CDK inhibitors. At the lowest concentration (9 nM) Dinaciclib signature was similar to that of 5-Fluorouracil (5-FU). Therefore, cell response to NU6140 was dominated by Aurora inhibition, which is in agreement with data by others. The CDK inhibition dominated only in a narrow concentration range. Its signature at the highest concentration suggested cell stress similar to that produced by heavy metals. In contrast, the dominant response to Dinaciclib was consistent with CDK inhibition, and the signature at lowest concentration suggested inhibited DNA synthesis, which is consistent with the activity of both Dinaciclib29 and 5-FU30.

AKT inhibitors were assessed. The Akt1 inhibitor A-674563 is known to inhibit PKA and Cdk2 with comparable potencies. A high similarity of A-674563 signatures to the on-target signature for CDK inhibitors (from 70 nM to 1.7 uM) was observed; at lower concentrations, no matching signature was located in the reference database. The signatures for a selective pan-Akt inhibitor GDC-0068 (Ipatasertib) at all tested concentrations (60 nM to 15 uM) had high-similarity to the invariant signature of AKT inhibitors (r>0.80). Therefore, the dominant effect of A-674563 was CDK inhibition, as compared to GDC-0068 whose dominant effect was AKT inhibition at all tested concentrations.

ERK inhibitors were also assessed. The signatures for ERK1/2 inhibitor GDC-0994 had a high similarity to the on-target signatures of ERK/MEK inhibitors at concentrations from 27 nM to 2.2 uM. The signatures at higher concentrations matched the invariant signature of HDAC inhibitors. Another ERK1/2 Ulixertinib (BVD-523) also produced high-similarity signatures to the on-target signature for ERK/MEK PKIs at concentrations from 27 nM to 0.74 uM. At higher concentrations, Ulixertinib TFAPs were similar to those of a prototypical mitochondria inhibitor, rotenone. These data indicate that cell responses to GDC-0994 and Ulixertinib were dominated by ERK/MEK inhibition in a broad range of concentrations, but these PKIs produced had off-target effects of non-kinase targets (HDAC inhibition vs. mitochondria malfunction).

Combined, these data demonstrated the unique capabilities of TFAP-based evaluation for the identification of kinase inhibitors' effects on kinases and non-kinase targets, as well as for determining the concentration ranges where those effects dominate.

The invariant PKI TFAP signatures permit the identification of novel kinase inhibitors with specified activities. In a study of the present methodology, the invariant signatures for AKT and CDK inhibitors were used to query a TFAP database containing over 30,000 signatures for diverse chemicals. Numerous high similarity signatures were retrieved. Literature searches showed that some of retrieved chemicals were known PKI inhibitors. For example, three known AKT inhibitors (paroxetine, ethanol, and DMSO) were retrieved and known CDK inhibitors harmine and Aflatoxin B were retrieved.

The methodology of the present disclosure thus enables: (1) assessment of polypharmacology (bioactivity profiles) of kinase inhibitors; (2) a systematic functional annotation of the human kinases in the context of vertical pathways; and (3) development of chemical probes to deorphanize dark kinases.

The observations of the present inventors indicate that the TFAP signature of a PKI changes with concentration, implying that different PKI activities shape cell response at different concentrations. At some concentrations, a PKI's signature matches the invariant signature for a particular kinase. By identifying the most similar invariant kinase signature(s), the inhibitory activity dominating cell response to a multi-kinase PKI at a given concentration can be determined, as well as the concentration ranges (specificity windows) for these dominant PKI activities.

In the development of the methodology of the present disclosure, the present inventors have found that the signature for a kinase-dead (KD) Akt mutant perfectly matched the common signature for chemical inhibitors. Dark kinases may be inactivated by use of appropriate KD mutants. Alternatively, RNAi or CRISPR/Cas9 techniques may be employed.

KD mutants for dark kinases can be generated by mutating the critical Lvs residue within the ATP-binding kinase domains, and bioinformatics techniques can be used to deduce the sequences of the KD mutants. These plasmids can be co-expressed in assay cells along with the FACTORIAL™ reporter plasmids, using appropriately optimized conditions. Such technique enables use of the obtained signatures to discover new chemical inhibitors for dark kinases.

TFAP datasets enable retrieval of the best-matching dark kinases' signatures. To validate the retrieved compounds as dark kinase inhibitors, proteomics-based kinase profiling assays may be employed. Retrieved compounds may predominantly be non-selective inhibitors for multiple kinases. These PKI's may be employed as scaffolds to synthesize high-selectivity chemical probes for dark kinases, utilizing iterative medicinal chemistry to develop inhibitor series, seeking at each step for the derivatives with extended specificity windows for a particular kinase. To assess inhibitors' selectivity, proteomics-based kinase profiling techniques may be employed.

PKI screening may employ any suitable cells, e.g., an HG19 clone of HepG2 (human liver hepatoma cell line, ATCC# HB-8065), a clone exhibiting elevated xenobiotic metabolizing activity due to a high level expression of CYP450 enzymes.

In profiling kinase activity in assay cells, endogenous kinases can be pulled down from cell lysates of the assay cells using beads with immobilized pan-kinase inhibitors, with isolated proteins resolved and analyzed by MS analysis.

The FACTORIAL™ system utilized in the method of the present disclosure comprises a library of reporter transcription units (RTUs), reporter constructs whose activities are assessed by quantitating the reporter RNAs. Each RTU has a TF-responsive promoter linked to a downstream reporter sequence. All RTUs have identical reporter sequences, “tagged” by a unique Hpa I restriction site at a distinct position within the RTUs. The RTU plasmids are transiently co-transfected into assay cells; subsequently, RTU transcripts are extracted from cell lysates and amplified by RT-PCR, using one pair of fluorescently labeled primers. Cleaving the PCR products by HpaI produces labeled DNA fragments of predetermined lengths. The labeled fragments are resolved by capillary electrophoresis (CE), and the RTUs' activity is assessed by profiling the CE bands. Owing to RTU homogeneity, all reporter RNAs are equally susceptible to variations of detection conditions (RNA degradation, amplification, etc.). As a result, the FACTORIAL™ system provides equal detection efficacy for each TF, thereby ensuring extraordinarily reproducible TF activity profiles.

The FACTORIAL™ assay may employ any suitable number of RTUs. In various embodiments, the assay may utilize 48 RTUs whose promoters contain one or multiple copies of TF binding sites, allowing detecting multiple TFs that regulate transcriptional responses to various stress stimuli, cytokines, and growth factors (including NFkB, AP-1, HIF-1a, p53, Xbp1, etc.).

In the method wherein HepG2 cells are employed, such cells can be transiently transfected with the FACTORIAL™ RTUs, plated into wells of a 24 well plate and incubated for 24 hours with evaluated PKIs in a 1% FBS culture medium. Each well can constitute a separate FACTORIAL™ assay producing one TFAP signature. Each screening set can contain evaluated PKIs at multiple concentrations, a negative control (the vehicle), and two positive controls (PKIs with well-defined signatures). To ensure the responsiveness of the assay, a standard series of inducers may be included in each screening batch as positive controls for individual TF endpoints, including TNFa (for NF-kB), estradiol (for ER), dexamethasone (for GR), rosiglitazone (for PPAR), etc.

Concerning the PKI concentration range, since the Kd values for the target kinases for most PKIs are <100 nM, a maximum concentration will be of 6 uM, to capture both the on-target and the off-target effects (usually detected at micromolar concentrations). Each PKI can be screened in serial dilutions, as for example six 1/3 serial dilutions, covering the range from 2 uM to 6 nM. Such concentration scale may be selected to be consistent with the one used for generating the reference TFAP database. Typically, the lowest concentrations of PKIs will produce a “null” TFAP signature (no TF responses). If a PKI still produces non-zero signatures at the lowest concentration (6 nM in this example), it may be additionally screened at further dilutions. Each PKI concentration can be evaluated by multiple assay runs, e.g., three independent FACTORIAL™ assays, to obtain a corresponding number of replicate TFAP signatures.

In providing the PKI TFAP signatures, differential PKI TFAPs can be calculated to show log-transform fold-changes of transcription factor activity in PKI-treated versus vehicle-treated cells, with the PKI TFAPs being presented as radiographs.

While the disclosure has been set out herein in reference to specific aspects, features and illustrative embodiments, it will be appreciated that the utility of the disclosure is not thus limited, but rather extends to and encompasses numerous other variations, modifications and alternative embodiments, as will suggest themselves to those of ordinary skill in the field of the present disclosure, based on the description herein. Correspondingly, the invention as hereinafter claimed is intended to be broadly construed and interpreted, as including all such variations, modifications and alternative embodiments, within its spirit and scope.

1. A method to identify perturbagens of particular biological processes and cell systems within living cells, said method comprising
exposing test cells to reference compounds from a group of known perturbagens of a particular biological process or a cell system within said cells;
evaluating the activity of transcription factors (TFs) within the exposed test cells resulting from said exposing to each reference compound;
identifying the consensus transcription factor activity profile (TFAP) for said group of reference compounds;
exposing test cells to an evaluated compound;
evaluating the activity of TFs within said test cells resulting from exposing the test cells to the evaluated compound,
calculating the similarity value of the TFAP of the evaluated compound to the consensus TFAP for the reference compounds, and
annotating the evaluated compound as a perturbagen of said biological process or said cell system if said similarity value exceeds a preset threshold value.

2. The method of claim 1, wherein said perturbagen is an inhibitor, an activator, or a disruptor of a biological process or cell system.

3. The method of claim 1, wherein said perturbagen is an inhibitor of mitochondria function, ubiquitin-proteasome-mediated protein degradation process, histone deacetylation, protein phosphorylation, protein dephosphorylation, lipid phosphorylation, or lipid dephosphorylation.

4. The method of claim 1, wherein said perturbagen is a DNA damaging agent, a cytoskeleton disruptor, or an inducer of proteotoxic shock.

5. The method of claim 1, wherein said perturbagen is an inhibitor of an Akt kinase, an mTOR kinase, a Mek kinase, a Raf kinase, a ERK kinase, an Aurora kinase, a cyclin dependent kinase, or a phosphodiesterase inhibitor.

6. The method of claim 1, wherein said group of known reference compounds comprises at least two compounds.

7. The method of claim 1, wherein said TF activity profile comprises activity values for at least 3 TFs.

8. The method of claim 7, wherein the TFs are selected from the following group: NF-kappaB, AP-1, myc, HIF-1a, p53, PXR, MTF-1, HSF-1, beta-catenin/TCF.

9. The method of claim 1, wherein TF activity is determined using an assay permitting quantitative assessment of TF activity.

10. The method of claim 9, wherein said assay is a DNA binding assay, a gel-shift assay, a reporter gene assay, or a transcription-based homogeneous reporter system assay.

11.-14. (canceled)

15. The method of claim 1, wherein said TFAP similarity value for TFAPs is a Pearson correlation coefficient or a Euclidean distance.

16. The method of claim 1, wherein said TFAP similarity threshold is >0.5; >0.6; >0.7; >0.8; >0.9; or >0.95.

17. The method of claim 1, wherein said consensus TFAP for the group of reference compounds is identified using cluster analysis of TFAPs for individual perturbagens.

18. The method of claim 1, wherein said evaluated compound is a chemical, a mix of chemicals, an RNA molecule, a DNA molecule, a peptide, a protein, or an antibody.

19. The method of claim 1, wherein said evaluated compound is a biological specimen.

20. (canceled)

21. The method of claim 1, wherein said test cells are exposed to different concentrations of reference perturbagens.

22. The method of claim 1, wherein said test cells are exposed to said reference compounds for a predetermined time.

23. The method of claim 22, wherein said predetermined time is more than 0.5 hrs, more than 1 hrs, more than 3 hrs, more than 12 hrs, more than 24 hrs, more than 48 hrs, or more than 72 hrs.

24. The method of claim 1, wherein said TFAP similarity value is calculated using a computer program.

25. The method of claim 1, wherein at least one of the method steps is a computer-implemented operation.
